(19) United States
(12) Patent Application Publication
(10) Pub. No.: US 2026/0029269 A1
(43) Pub. Date: Jan. 29, 2026

(54) RADIO-FREQUENCY IDENTIFICATION (RFID) CAPACITANCE LIQUID MEASUREMENT SENSOR TAG SYSTEM

(71) Applicant: Daniel Knotts, Atlanta, GA (US)

(72) Inventor: Daniel Knotts, Atlanta, GA (US)

(21) Appl. No.: 19/349,930

(22) Filed: Oct. 3, 2025

Related U.S. Application Data
(63) Continuation-in-part of application No. 17/739,998, filed on May 9, 2022, now Pat. No. 12,020,105.
(60) Provisional application No. 63/186,045, filed on May 7, 2021.
(60) Provisional application No. 63/702,689, filed on Oct. 3, 2024.

(51) Int. Cl. G01F 23/263; G01N 27/22; G01N 33/24

(52) U.S. Cl. CPC G01F 23/266; G01N 27/223; G01N 33/246

(57) ABSTRACT

A liquid level detection system may comprise a sensor tag insertable into a container. The sensor tag may include a capacitance sensor, a chip, and an RFID inlay encapsulated within a rigid material. The sensor tag may be provided in different lengths with unique identifiers such as colors, numbers, or symbols. The sensor tag may include a weighting element at its bottom portion and RFID components positioned near the top. Power may be derived from the RFID antenna or from an internal battery. The system may function by measuring changes in capacitance as liquid levels vary, with the chip processing the data and the RFID inlay transmitting to external readers. The system may be adapted for various applications including beverage inventory management and agricultural soil monitoring, providing accurate, automated monitoring capabilities for liquid levels or soil moisture conditions.

### PRIORITY DOCUMENTS

This application is a Continuation-In-Part of U.S. patent application Ser. No. 17/739,998 filed May 9, 2022, which issued on Jun. 25, 2024 as U.S. Pat. No. 12,020,105, which claims priority to U.S. Provisional Patent Application No. 63/186,045 filed May 7, 2021. This application is a non-provisional conversion of U.S. Provisional application 63/702,689 filed Oct. 3, 2024. All are hereby incorporated by reference in their entirety.

It may be understood that each of the referenced applications may be applicable to the concepts and embodiments disclosed herein, even if such concepts and embodiments are disclosed in the referenced applications with different limitations and configurations and described using different examples and terminology.

### FIELD

The present disclosure relates to systems using radio-frequency identification (RFID) tag antenna-based sensors. More particularly, the disclosure relates to capacitance-based liquid level detection systems for inventory management applications. The systems may be employed for various applications, such as liquid level detection, beverage inventory management, agricultural soil monitoring, and similar sensing applications.

### BACKGROUND

The following background information may be provided to assist the reader in understanding the technology described below. The background information may include concepts, terms, or other material that may be known to those skilled in the art but that may be helpful in understanding the claimed subject matter. The background information should not be construed as an admission that any of the material described is prior art to the claims in this application.

Various industries face challenges relating to accurate liquid level detection and inventory management. The hospitality industry encounters significant problems relating to loss of product, particularly with expensive beverages such as wine and liquor. These issues may be exacerbated by factors including employee theft, over-pouring, and ineffective tracking of inventory lifecycles.

Traditional approaches for liquid level detection may be susceptible to human error or may employ complex electronics that are not cost-effective for large-scale implementation. Visual inspection methods may be prone to inaccuracy. Electronic systems may be cumbersome, difficult to adopt, or challenging to integrate into existing operations.

Agricultural applications similarly face challenges in monitoring soil conditions. Conventional soil moisture sensors may have limited penetration depth, require active battery power, or may not provide long-term reliability. These limitations may restrict their effectiveness in providing accurate subsurface monitoring data.

There may be a need for improved liquid level detection systems that can provide accurate, cost-effective, and scalable solutions for various applications including beverage inventory management and agricultural monitoring.

### BRIEF OVERVIEW

The following overview may be provided to introduce concepts that may be described in more detail below. This overview may not be intended to identify essential features of the claimed subject matter, nor may it be intended to limit the scope of the claimed subject matter.

Various embodiments may be directed to different combinations and sub-combinations of the features described in the detailed description. The scope of the claimed subject matter may not be limited to the particular combinations described herein, as other combinations and variations may be possible.

Additional aspects of the disclosure may be set forth in the description which follows. The advantages of the disclosure may be realized through the elements and combinations particularly pointed out in the appended claims. The foregoing general description and the following detailed description may be explanatory only and may not be restrictive of the disclosure as claimed.

A liquid level detection system may comprise a sensor tag insertable into a container. The sensor tag may comprise a capacitance sensor, a chip, and a radio-frequency identification (RFID) inlay. The capacitance sensor, the chip, and the RFID inlay may be encapsulated within the sensor tag. The sensor tag may be fabricated from at least one of a rigid material, semi-rigid material, or flexible material. The sensor tag may be encapsulated with a material comprising BPA-free plastic, laminated sheets, or other encapsulating material. In one or more embodiments, the sensor tag may be fully submerged, such that all portions of the sensor tag is submerged in the liquid or environment. The sensor tag may be provided in a plurality of lengths. Each length of the plurality of lengths may be associated with a unique identifier. The unique identifier may comprise at least one of a color, a number, or a symbol.

In one or more embodiments, the sensor tag may further comprise a weighting element at a bottom portion of the sensor tag. The RFID inlay may be positioned near a top portion of the sensor tag. Power for the sensor tag may be derived from the RFID inlay. The sensor tag may further comprise an internal battery. The internal battery may be encapsulated within the sensor tag. The capacitance sensor may be configured to detect changes in capacitance as a liquid level in the container varies. The chip may be configured to process the changes in capacitance detected by the capacitance sensor. The RFID inlay may be configured to transmit data processed by the chip to an external reader device. The sensor tag may further comprise a flotation element. In one or more embodiments, embodiments, the sensor tag may be placed in a container and be designed in such a way that it does not come back out of the container. With slight tension between the material and the mouth of the container, it may be easy for a user to place a stick inside a bottle and still be able to pour drinks without the stick coming out. In one or more embodiments, the sensor tag may feature logo marks, brand information, trademarks, slogans for associated beverages and brands.

A method for liquid level detection may comprise inserting a sensor tag into a container. The sensor tag may comprise a capacitance sensor, a chip, and a radio-frequency identification (RFID) inlay encapsulated within the sensor tag. The method may include detecting changes in capacitance as a liquid level in the container varies using the capacitance sensor. The method may include processing the detected changes in capacitance using the chip. The method may include transmitting data processed by the chip to an external reader device using the RFID inlay. In one or more embodiments, the external reader device may be a smartphone, laptop, smart watch, wearable device, or computer.

A liquid inventory management system may comprise a plurality of sensor tags. Each sensor tag may be insertable into a container and may comprise a capacitance sensor, a chip, and a radio-frequency identification (RFID) inlay encapsulated within the sensor tag. The system may include at least one RFID reader device configured to receive data from the plurality of sensor tags. The system may include a data processing component configured to analyze the received data and generate inventory status reports.

The system may be adapted for agricultural applications. The sensor tag may be configured for insertion into soil or growing media. The system may be configured to monitor soil moisture levels, temperature, and potentially pH levels. The sensor tag may be designed for deep soil penetration to depths of six inches or more below the surface. The system may provide battery-free operation through passive wireless communication protocols.

The wireless communication capabilities of the liquid measurement sensor tag system may encompass multiple transmission technologies to enable comprehensive data transfer and remote monitoring functionality. The RFID inlay may be configured to support Near Field Communication (NFC) protocols for short-range data transmission to compatible mobile devices and readers. The wireless communication module may utilize Bluetooth Low Energy (BLE) technology to establish connections with smartphones, tablets, and other portable electronic devices within a moderate range. The system may incorporate 5G cellular communication capabilities to enable long-range data transmission and real-time connectivity to cloud-based inventory management platforms. Wi-Fi connectivity may be integrated into the sensor tag system to facilitate wireless data transmission over existing wireless network infrastructure.

The transmission technologies may operate independently or in combination to provide redundant communication pathways for enhanced system reliability. The NFC functionality may enable tap-to-read operations where users may simply bring an NFC-enabled device into proximity with the sensor tag to retrieve liquid level data. The BLE communication may provide continuous or periodic data streaming to paired devices within the operational range. The 5G connectivity may enable real-time data synchronization with remote servers and cloud-based analytics platforms for comprehensive inventory management across multiple locations. The Wi-Fi capabilities may allow the sensor tags to connect directly to local area networks for seamless integration with existing business management systems.

In one or more embodiments, the liquid measurement system may be implemented in an agricultural environment. In this embodiment, the liquid measurement system may function as a soil monitoring system may comprise a sensor device insertable into soil. The sensor device may comprise a capacitance sensor configured to detect soil moisture levels. The sensor device may comprise a processing chip. The sensor device may comprise a wireless communication module. The capacitance sensor, the processing chip, and the wireless communication module may be encapsulated within a rigid housing configured for soil penetration.

A method for liquid level detection may comprise inserting a sensor tag into a container. The sensor tag may comprise a capacitance sensor, a chip, and a radio-frequency identification (RFID) inlay encapsulated within the sensor tag. The method may comprise detecting changes in capacitance as a liquid level in the container varies using the capacitance sensor. The method may comprise processing the detected changes in capacitance using the chip. The method may comprise transmitting data processed by the chip to an external reader device using the RFID inlay.

A liquid inventory management system may comprise a plurality of sensor tags. Each sensor tag may be insertable into a container and may comprise a capacitance sensor, a chip, and a radio-frequency identification (RFID) inlay encapsulated within the sensor tag. The system may comprise at least one RFID reader device configured to receive data from the plurality of sensor tags. The system may comprise a data processing component configured to analyze the received data and generate inventory status reports.

The liquid inventory management system may comprise a sensor tag delivery and packaging system that may provide efficient storage and dispensing solutions for hospitality environments. The system may comprise tube or canister packaging that enables alphabetized organization and modular arrangement without requiring additional shelving or storage hardware. The tubes may be arranged vertically or horizontally to optimize space utilization in inventory rooms, offices, kitchen areas, and industrial settings.

The packaging system may feature clear markings, colors, and identification information to enable quick stick selection. A QR code may be displayed on the tube graphics to facilitate rapid product reordering through scanning functionality. Transparent areas of the tube may allow visual inspection of stick quantities without opening the container. The transparent sections may serve as quick visual indicators for inventory levels.

The tube design may incorporate a hinge cap or flip cap at the top portion to enable one-handed operation. The cap mechanism may allow users to open the tube, retrieve sticks, and close the tube using a single hand. A magnetic element may be positioned at the bottom of the tube to secure the tubes firmly in place when positioned on metal surfaces. The magnetic attachment may be particularly effective on metal tables and surfaces commonly found in inventory rooms, offices, kitchen areas, and industrial settings.

The tube system may accommodate multiple stick sizes through a standardized tube configuration. Foam spacers may be inserted into the tubes to accommodate different stick lengths while maintaining uniform tube dimensions. The foam spacer system may enable inventory standardization across various stick configurations.

Alternative packaging configurations may include cartridge-style dispensers. The cartridge system may incorporate gravity-fed or spring-loaded mechanisms to facilitate stick retrieval. The cartridge may feature dispensing slots that provide individual stick access. The cartridge design may enable automated stick dispensing through mechanical feeding systems.

### BRIEF DESCRIPTION OF THE DRAWINGS

The accompanying drawings may be incorporated in and may constitute a part of this specification. The drawings may illustrate various embodiments of the present disclosure. The drawings may contain representations of various trademarks and copyrights owned by the Applicants. The drawings may contain other marks owned by third parties and may be used for illustrative purposes only. All rights to various trademarks and copyrights represented herein, except those belonging to their respective owners, may be vested in and the property of the Applicants.

The drawings may contain text or captions that may explain certain embodiments of the present disclosure. This text may be included for illustrative, non-limiting, explanatory purposes of certain embodiments detailed in the present disclosure.

Furthermore, it may be understood that various modifications and changes can be made without departing from the scope of the present disclosure. The drawings may be regarded in an illustrative rather than a restrictive sense, and all such modifications may be intended to be included within the scope of the present disclosure.

FIG. 1A shows an RFID capacitance liquid measurement sensor tag system configuration in accordance with an embodiment of the invention.

FIG. 1B illustrates multiple configurations of RFID chips and capacitance sensors within sensor tag systems of the RFID capacitance liquid measurement sensor tag system configuration in accordance with an embodiment of the invention.

FIG. 1C depicts an RFID capacitance liquid measurement sensor tag system configured for liquid measurement applications in accordance with an embodiment of the invention.

FIG. 1D depicts an RFID capacitance liquid measurement sensor tag system configured for liquid measurement applications in accordance with an embodiment of the invention.

FIG. 2A shows various components of the RFID capacitance liquid measurement sensor tag system configured for liquid measurement applications in accordance with an embodiment of the invention.

FIG. 2B illustrates bottle configurations for bottles utilizing the RFID capacitance liquid measurement sensor tag system configured for liquid measurement applications in accordance with an embodiment of the invention.

FIG. 2C depicts a sensor tag fully submerged in liquid within a container for an RFID capacitance liquid measurement sensor tag system configured for liquid measurement applications in accordance with an embodiment of the invention.

FIG. 3A shows a handheld reader device scanning an RFID capacitance liquid measurement sensor tag system configured for liquid measurement applications in accordance with an embodiment of the invention.

FIG. 3B depicts multiple sensor tags being read by a handheld device with extended range capabilities for the RFID capacitance liquid measurement sensor tag system configured for liquid measurement applications in accordance with an embodiment of the invention.

FIG. 4A illustrates a process for determining sensor tag selection with scanned containers for the RFID capacitance liquid measurement sensor tag system configured for liquid measurement applications in accordance with an embodiment of the invention.

FIG. 4B shows mobile device interfaces with color-coded identification systems for the RFID capacitance liquid measurement sensor tag system configured for liquid measurement applications in accordance with an embodiment of the invention.

FIG. 4C depicts color-coded and numbered liquid level detection sensor tags for the RFID capacitance liquid measurement sensor tag system configured for liquid measurement applications in accordance with an embodiment of the invention.

FIG. 4D shows hard sensor tag units with identification features for the RFID capacitance liquid measurement sensor tag system configured for liquid measurement applications in accordance with an embodiment of the invention.

FIG. 4E and FIG. 4F illustrates different types and views of RFID sensor tags for the RFID capacitance liquid measurement sensor tag system configured for liquid measurement applications in accordance with an embodiment of the invention.

FIG. 5A shows a liquid level detection system implemented in a glass container for the RFID capacitance liquid measurement sensor tag system configured for liquid measurement applications in accordance with an embodiment of the invention.

FIG. 5B shows a liquid level detection system implemented in a glass container for the RFID capacitance liquid measurement sensor tag system configured for liquid measurement applications in accordance with an embodiment of the invention.

FIG. 5C illustrates a sensor tag unit in a beverage bottle for the RFID capacitance liquid measurement sensor tag system configured for liquid measurement applications in accordance with an embodiment of the invention.

FIG. 5D shows compatibility with existing pour spouts and bottle configurations for the RFID capacitance liquid measurement sensor tag system configured for liquid measurement applications in accordance with an embodiment of the invention.

FIG. 5E illustrates a sensor tag unit in a beverage bottle for the RFID capacitance liquid measurement sensor tag system configured for liquid measurement applications in accordance with an embodiment of the invention.

FIG. 6A shows storage and organization systems for the RFID capacitance liquid measurement sensor tag system configured for liquid measurement applications in accordance with an embodiment of the invention.

FIG. 6B depicts a sensor tag containing granular material for the RFID capacitance liquid measurement sensor tag system configured for liquid measurement applications in accordance with an embodiment of the invention.

FIG. 6C illustrates transparent container storage systems for the RFID capacitance liquid measurement sensor tag system configured for liquid measurement applications in accordance with an embodiment of the invention.

FIG. 7A shows a user platform dashboard interface configuration for the RFID capacitance liquid measurement sensor tag system configured for liquid measurement applications in accordance with an embodiment of the invention.

FIG. 7B illustrates a dashboard interface with analytics and reporting features for the RFID capacitance liquid measurement sensor tag system configured for liquid measurement applications in accordance with an embodiment of the invention.

FIG. 7C illustrates a dashboard interface with analytics and reporting features for the RFID capacitance liquid measurement sensor tag system configured for liquid measurement applications in accordance with an embodiment of the invention.

FIG. 7D shows a laptop computer displaying dashboard interface for the RFID capacitance liquid measurement sensor tag system configured for liquid measurement applications in accordance with an embodiment of the invention.

FIG. 7E illustrates sensor data visualization with graphs and terminal output for the RFID capacitance liquid measurement sensor tag system configured for liquid measurement applications in accordance with an embodiment of the invention.

FIG. 7F shows integrated dashboard and mobile ordering interface for the RFID capacitance liquid measurement sensor tag system configured for liquid measurement applications in accordance with an embodiment of the invention.

FIG. 8A depicts mobile application screens for inventory management for the RFID capacitance liquid measurement sensor tag system configured for liquid measurement applications in accordance with an embodiment of the invention.

FIG. 8B shows mobile interface screens for product details and variance reporting for the RFID capacitance liquid measurement sensor tag system configured for liquid measurement applications in accordance with an embodiment of the invention.

FIG. 8C illustrates mobile dashboard interfaces with inventory analytics for the RFID capacitance liquid measurement sensor tag system configured for liquid measurement applications in accordance with an embodiment of the invention.

FIG. 8D depicts mobile shopping and ordering interfaces for the RFID capacitance liquid measurement sensor tag system configured for liquid measurement applications in accordance with an embodiment of the invention.

FIG. 8E shows a barcode scanning interface for inventory applications for the RFID capacitance liquid measurement sensor tag system configured for liquid measurement applications in accordance with an embodiment of the invention.

FIG. 8F illustrates a mobile application home screen for the RFID capacitance liquid measurement sensor tag system configured for liquid measurement applications in accordance with an embodiment of the invention.

FIG. 8G depicts a welcome screen with NFC technology instructions for the RFID capacitance liquid measurement sensor tag system configured for liquid measurement applications in accordance with an embodiment of the invention.

FIG. 9A shows a RFID capacitance liquid measurement sensor tag system configured for liquid measurement applications in accordance with an embodiment of the invention for drone system for agricultural field monitoring.

FIG. 9B illustrates a RFID capacitance liquid measurement sensor tag system configured for liquid measurement applications in accordance with an embodiment of the invention for ground-based drone configuration in an agricultural setting.

FIG. 9C depicts a RFID capacitance liquid measurement sensor tag system configured for liquid measurement applications in accordance with an embodiment of the invention showing soil insertion tools for sensor deployment.

FIG. 9D shows a RFID capacitance liquid measurement sensor tag system configured for liquid measurement applications in accordance with an embodiment of the invention for oil moisture monitoring interface with field mapping and data visualization.

FIG. 9E shows a mobile interface for a RFID capacitance liquid measurement sensor tag system configured for liquid measurement applications in accordance with an embodiment of the invention for displaying soil moisture data and field mapping with data visualization interfaces.

### DETAILED DESCRIPTION

The present disclosure may include many aspects and features. While many aspects and features may relate to liquid level detection systems, embodiments of the present disclosure may not be limited to use only in this context. The present disclosure may be understood more readily by reference to the following detailed description and the examples included therein.

It may be understood that the articles, systems, apparatuses, and methods described herein may not be limited to specific manufacturing methods unless otherwise specified, or to particular materials unless otherwise specified. It may also be understood that the terminology used herein may be for the purpose of describing particular aspects only and may not be intended to be limiting. Any methods and materials similar or equivalent to those described herein may be used in the practice or testing of the present disclosure.

The terminology used herein may be for the purpose of describing particular aspects only and may not be intended to be limiting. As used in the specification and in the claims, the term “comprising” may include the aspects “consisting of” and “consisting essentially of.” Unless defined otherwise, all technical and scientific terms used herein may have the same meaning as commonly understood by one of ordinary skill in the art to which this disclosure belongs.

As used in the specification and the appended claims, the singular forms “a,” “an” and “the” may include plural referents unless the context clearly dictates otherwise. Ranges may be expressed herein as from one particular value, and/or to another particular value. When such a range may be expressed, another aspect may include from the one particular value and/or to the other particular value. When values may be expressed as approximations, by use of the antecedent ‘about,’ it may be understood that the particular value may form another aspect.

As used herein, the terms “about” and “at or about” may mean that the amount or value in question may be the value designated some other value approximately or about the same. It may be generally understood that it may be the nominal value indicated ±10% variation unless otherwise indicated or inferred. The term may be intended to convey that similar values may promote equivalent results or effects recited in the claims.

The terms “optional” or “optionally” may mean that the subsequently described event or circumstance may or may not occur, and that the description may include instances where said event or circumstance occurs and instances where it does not. Unless otherwise expressly stated, it may be in no way intended that any method set forth herein be construed as requiring that its steps be performed in a specific order.

It may be understood that when combinations, subsets, interactions, groups, etc. of materials may be disclosed that while specific reference of each various individual and collective combinations and permutation of these materials may not be explicitly disclosed, each may be specifically contemplated and described herein. If a class of materials A, B, and C may be disclosed as well as a class of materials D, E, and F and an example of a combination material, A-D may be disclosed, then even if each may not be individually recited each may be individually and collectively contemplated.

It may be understood that the apparatuses and systems disclosed herein may have certain functions. There may be a variety of structures that can perform the same function that may be related to the disclosed structures, and that these structures may typically achieve the same result.

Referring now to the drawings, examples of liquid level detection systems and concepts thereof may be described. The present disclosure may relate to a liquid level detection system for containers. The system may comprise a sensor tag that can be inserted into a container. The sensor tag may include a capacitance sensor, a chip, and an RFID inlay. These components may be fully encapsulated or sealed within plastic, silicone, rubber or another liquid safe material.

The sensor tag may be in a form factor that may permit the sensor tag to be inserted into the mouth of a container and remain upright in the container. This configuration may permit the RFID antenna and chip to have airspace surrounding it enabling accurate liquid level measurement. The sensor tag may be fabricated from a rigid material. The encapsulation material used for the sensor tag may be similar to materials commonly used in pour spouts or stoppers, such as BPA-free plastics.

The sensor tag may be provided in different lengths to accommodate various container sizes. To distinguish between different sensor tag lengths, the sensor tags may be color-coded, numbered, or marked with symbols. The sensor tag may include a weighting element at its bottom portion. This weighting element may help ensure the sensor tag remains properly oriented within the container when filled with liquid.

The RFID chip and antenna of the sensor tag may be positioned near the top portion of the sensor tag. This configuration may optimize readability of the RFID components by external reader devices, as the top portion may be more likely to remain above the liquid level in the container. Power for the sensor tag's components may be supplied through multiple means. Power may be derived from the RFID antenna, potentially eliminating the need for an internal battery. The sensor tag may incorporate an internal, encapsulated battery. This battery-powered configuration may provide extended operational range or enable additional features such as illumination of the sensor tag within the container.

In one or more embodiments, the liquid level detection system may comprise a sensor tag 100, wherein the sensor comprises a chip 110, a capacitance sensor 115, and a HarvestComm Unit (HCU) 105. A HarvestComm Unit (HCU) is configured to harvest wireless energy and communicate over one or more of RFID, BLE (Bluetooth low energy), ambient IoT, Wi-Fi, 5G, mesh networking, zigbee, z-wave, NFC, cellular, ultra-wideband, ANT, and other capable emerging technology protocols. In one or more embodiments, the liquid level detection system may include a processing component 110 comprising at least one of a microcontroller, application-specific integrated circuit (ASIC), system-on-chip (SoC), field programmable gate array (FPGA), or digital signal processor (DSP). This may be embodied as a sensecore processor, a datafusion engine, a nanologic unit, a low-power ARM cortex-M chip, RISC-V core, embedded microchip, edgecompute node, embedded AI accelerator, TinyML chip, neuromorphic process or the likeness thereof.

The liquid level detection system may function by measuring changes in capacitance as the liquid level in the container varies. The capacitance sensor within the sensor tag may detect these changes, which can then be processed by the chip and transmitted via the RFID transmitter. The capacitance value may be either processed by a sensor tag chip or communicated to a software application running on a phone, PC, cloud, or host computer to perform the translation of capacitance reading into liquid measurement specific to that bottle size and shape based on the type of liquid being measured.

The sensor tag may incorporate flotation capabilities. This feature may allow the transmitter portion of the sensor tag to remain above the liquid level within the container, potentially improving signal transmission to external reader devices. The liquid level detection system may incorporate a bobber wherein the liquid level may be measured by the distance from a floating sensor to a fixed reference point. The stick sensor tag or hard sensor tag should remain rigid and not float to maintain accurate capacitance readings. The hard sensor tag or stick sensor tag may remain fixed as to change the height of the liquid on the stick may negatively affect the capacitance reading.

The liquid level detection system may provide inventory control capabilities for containers. By monitoring the liquid level within containers, the system may enable more efficient management of liquid inventories across various applications. The sensor tag may include a capacitance sensor, a chip, and an RFID inlay. These components may be fully sealed in plastic, encapsulated within a plastic casing, a metal casing, or other material casing conducive to the proper enablement of accurate liquid detection

In one or more embodiments of the a RFID capacitance liquid measurement sensor tag system configured for liquid measurement applications, the sensor tag may be embodied as a rigid sensor tag, hard sensor tag, or stick encased or constructed from materials including but not limited to glass, foam, rubber, silicone, ceramic, PVC, wood or natural fiber, synthetic fiber, metal, and other materials. The sensor tag may be fabricated from a rigid material. The encapsulation material used for the sensor tag may be similar to materials commonly used in pour spouts or stoppers, such as BPA-free plastics.

In one or more embodiments, the sensor tag and associated components of the liquid measurement sensor tag system 220 may be made my laminating an RFID inlay, a chip, an a capacitive strip sensor in a manner that the laminate is a thin film. In these embodiments, the sensor tag 100 is not necessarily rigid. In one or more embodiments, it may be semi-rigid. In one or more embodiments, it is flexible and can conform to the shape of the container. In one or more embodiments, the sensor tag and associated components may be configured to bend, flex, and mimic the shape of the container while still being operable.

In one or more embodiments, the liquid measurement sensor tag system 220 may be configured to operate while fully submerged in liquid. The sensor tag may maintain functionality when completely surrounded by liquid within the neck of a container. The liquid measurement sensor tag system 220 may be configured to resist alcohol and chemical exposure while remaining fully operational during submersion. The encapsulation material of the liquid measurement sensor tag system 220 may be fabricated from transparent or opaque plastic materials.

In one or more embodiments, the sensor tag may be provided in different lengths to accommodate various container sizes. To distinguish between different sensor tag lengths, the sensor tags may be color-coded, numbered, or marked with symbols. The sensor tag may include a weighting element at its bottom portion. This weighting element may help ensure the sensor tag remains properly oriented within the container when filled with liquid.

In one or more embodiments, the RFID chip 105 and antenna of the sensor tag 100 may be positioned near the top portion of the sensor tag. This configuration may optimize readability of the RFID components by external reader devices, as the top portion may be more likely to remain above the liquid level in the container. Power for the sensor tag's components may be supplied through multiple means. Power may be derived from the RFID antenna. The sensor tag may incorporate an internal, encapsulated battery. This battery-powered configuration may provide extended operational range or enable additional features such as illumination of the sensor tag within the container. A battery may be configured to allow the sensor tag to retain data on the chip and provide processing to run applications similar to on-board capacitance translation into fluid level readings.

In one or more embodiments, the liquid level detection system 220 may function by measuring changes in capacitance as the liquid level in the container varies. The capacitance sensor within the sensor tag may detect these changes, which can then be processed by the chip and transmitted via the RFID inlay. The sensor tag may incorporate flotation capabilities. This feature may allow the transmitter portion of the sensor tag to remain above the liquid level within the container, potentially improving signal transmission to external reader devices.

In one or more embodiments, the sensor tag 100 may be designed to be completely sealed within an encapsulation material. This encapsulation may protect the internal components from exposure to liquids or other potentially damaging substances. The encapsulation material may be a BPA-free plastic. This material choice may ensure compatibility with a wide range of liquid contents and may comply with safety regulations for food and beverage containers. The sealed inlay may prevent exposure to liquids, and also ensure that the liquid may not be contaminated by any part of the electronics, potential battery, or metal sensors as part of the inlay. Totally encapsulating may ensure that only food-safe materials come into contact with the customer's liquid inventory.

In one or more embodiments, the sensor tag 100 may be manufactured in a variety of lengths. Each length may correspond to a specific container size or volume range. This variety may allow for accurate liquid level detection across a diverse range of container types and sizes. To facilitate easy identification, each sensor tag length may be associated with a unique identifier. These identifiers may include different colors, numbers, or symbols applied to the exterior of the sensor tag.

In one or more embodiments, a weighting element at the bottom of the sensor tag 100 may be designed to counteract any buoyancy effects from the encapsulation material or internal air pockets. This weighting may help maintain the sensor tag in a vertical orientation within the container. The sensor tag may incorporate a flotation element near its top portion. This flotation element may be designed to keep the RFID components above the liquid level, even when the container may be full.

The power source for the sensor tag may vary depending on the specific application requirements. The sensor tag may operate solely on power derived from the RFID antenna through inductive coupling with an external reader device. The sensor tag may include an internal battery. This battery may be fully encapsulated within the sensor tag to prevent exposure to liquids. The battery-powered configuration may allow for extended operational range or additional features.

The chip within the sensor tag may be programmed to interpret the capacitance measurements from the sensor. It may convert these measurements into meaningful liquid level data that can be transmitted via the RFID inlay. The RFID inlay may be configured to transmit the liquid level data to external reader devices. This transmission may occur periodically or in response to interrogation signals from the reader devices.

The liquid level detection system may be compatible with various types of RFID reader devices. These may include handheld readers, fixed readers mounted on shelves or doorways, or mobile readers integrated into inventory management systems. The system may be designed to support multiple sensor tags within a given area. This may allow for simultaneous monitoring of numerous containers in a storage or retail environment.

The liquid level detection system may also store data on the stick itself for transmission to the reader when queried. This can allow individual pour events to be recorded, and transmitted for analysis when most convenient. A device not having any internal memory can only provide one measurement taken at the time of reading. If that period may be a week, no additional detail of what happened during that intervening week may be available. Having memory in the liquid level detection system may add additional capabilities allowing for recording of multiple periodic measurements as needed.

The system may include software components for data analysis and inventory management. These components may process the liquid level data from multiple sensor tags to provide comprehensive inventory status reports. The system may be configured to generate alerts or notifications based on predefined liquid level thresholds. These alerts may help users identify containers that require refilling or replacement.

Furthermore, the liquid level detection system 220 may be integrated with inventory management software. The inventory management software may comprise a database of liquor product SKUs and their characteristics. The software may interpret the capacitance measurements to determine liquid levels, volumes, or percentages remaining in the container. The system may connect to distributor inventory databases to enable automated reordering when inventory levels may be low. The software may provide a user interface for device pairing, readings, and inventory management.

Moreover, the liquid level detection system 220 may in one or more embodiments utilizing RFID and other potential transmitting technologies, may support multiple sensor tags being scanned and/or read simultaneously within a given area. Using fixed readers and/or antennas that can be mounted throughout an environment may provide for close to real-time inventory counting. This may allow for simultaneous monitoring of numerous containers in a storage or retail environment.

The ability to read multiple sensor tags with fixed readers may provide the ability to have real-time inventory status as well as the ability to measure pours, wherein the pours may be calculated by processing the difference in fluid level prior to pour and fluid level after the pour. The liquid level detection system may be configured in this manner to measure pours and inventor by monitoring specific server activity. Using fixed readers, or using a battery powered sensor tag with memory may enable real-time reads. This may improve inventory management as inventory status may only be available when the sensor tag may be read or updated.

The liquid level detection system may be applicable in various industries and settings. These may include beverage service establishments, chemical storage facilities, agricultural, cosmetic, pharmaceutical, medical, oil, fuel management systems, and household applications.

The sensor tag may incorporate a capacitance sensor for detecting changes in the dielectric properties of its surroundings. As the liquid level in the container changes, it may alter the capacitance measured by the sensor. The liquid level detection system may provide for other uses of sensing capabilities such as detecting strain, pressure, and sample chemical properties like pH, acidity, alcohol content, and the likeness thereof.

Strain and temperature may be measured by a resistive sensor which may be adapted and added to one or more hard sensor tags. The liquid level detection system may comprise one or more elements with sensing capabilities including but not limited to capacitance sensors and resistance sensors adapted to measure fluid level, temperature, shake, or other properties of the container.

The chip within the sensor tag may be programmed to interpret the capacitance measurements from the sensor. It may convert these measurements into meaningful liquid level data that can be transmitted via the RFID inlay. The RFID inlay may be configured to transmit the liquid level data to external reader devices. This transmission may occur periodically or in response to interrogation signals from the reader devices.

The wireless communication capabilities of the liquid measurement sensor tag system may encompass multiple transmission technologies to enable comprehensive data transfer and remote monitoring functionality. The RFID inlay may be configured to support Near Field Communication (NFC) protocols for short-range data transmission to compatible mobile devices and readers. The wireless communication module may utilize Bluetooth Low Energy (BLE) technology to establish connections with smartphones, tablets, and other portable electronic devices within a moderate range. The system may incorporate 5G cellular communication capabilities to enable long-range data transmission and real-time connectivity to cloud-based inventory management platforms. Wi-Fi connectivity may be integrated into the sensor tag system to facilitate wireless data transmission over existing wireless network infrastructure.

The transmission technologies may operate independently or in combination to provide redundant communication pathways for enhanced system reliability. The NFC functionality may enable tap-to-read operations where users may simply bring an NFC-enabled device into proximity with the sensor tag to retrieve liquid level data. The BLE communication may provide continuous or periodic data streaming to paired devices within the operational range. The 5G connectivity may enable real-time data synchronization with remote servers and cloud-based analytics platforms for comprehensive inventory management across multiple locations. The Wi-Fi capabilities may allow the sensor tags to connect directly to local area networks for seamless integration with existing business management systems.

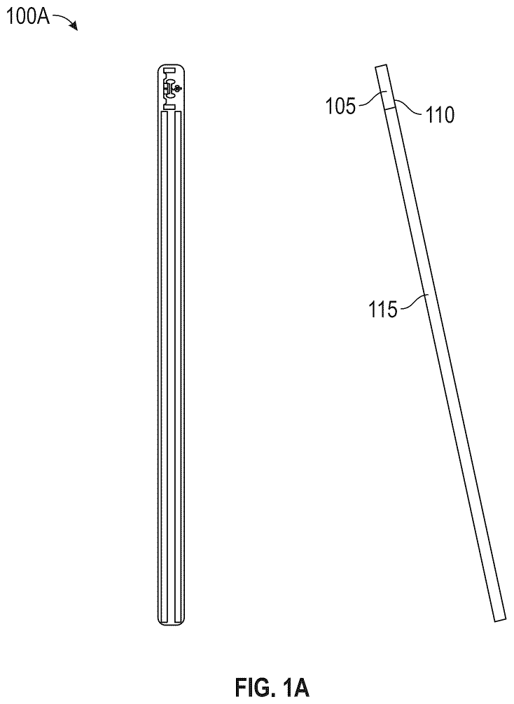

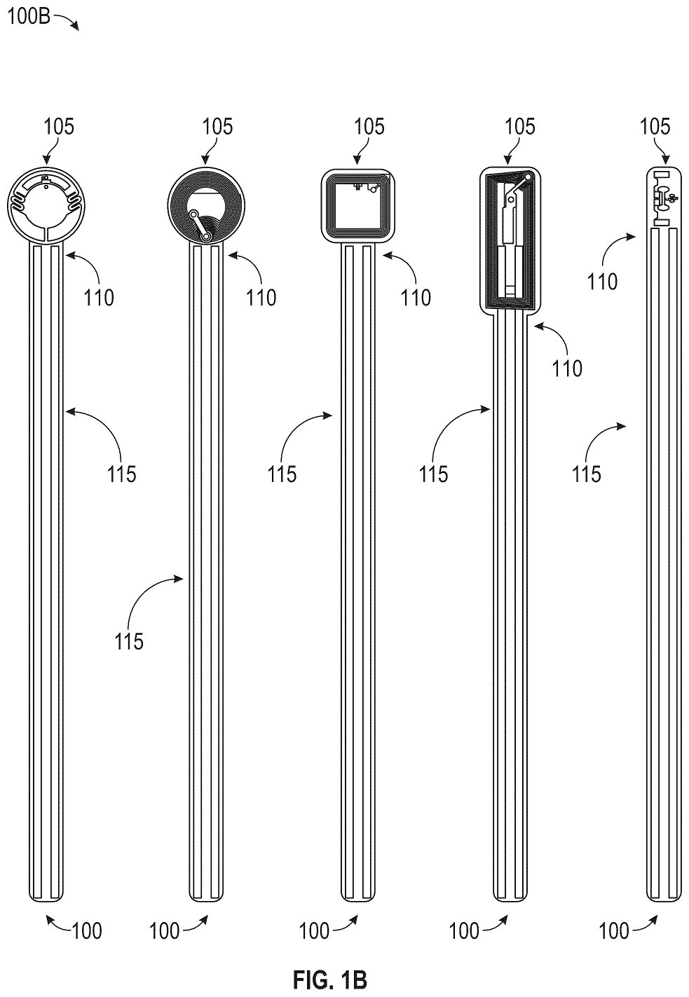

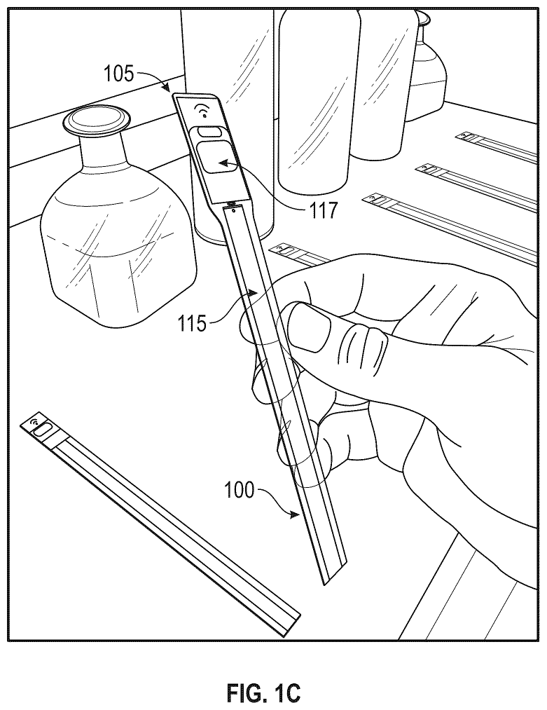

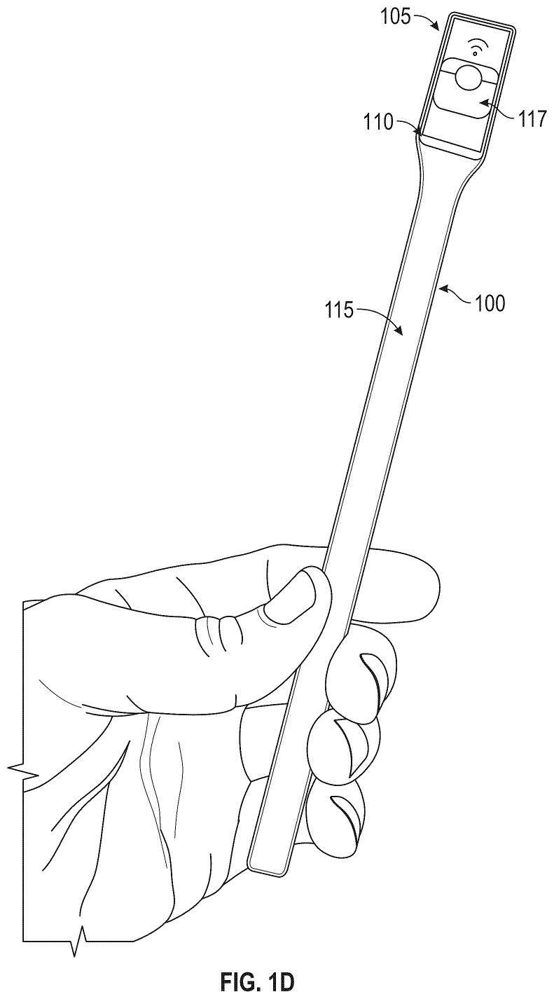

With reference to FIG. 1A, an example configuration of a radio-frequency identification (RFID) capacitance liquid measurement sensor tag system 100A may be shown. The system 100A may comprise a sensor tag with a capacitance sensor, RFID components, and a chip integrated into a single elongated device. The sensor tag may be designed for insertion into containers to measure liquid levels.

The figure shows two views of a sensor tag device of a liquid level detection system 220, labeled as 100. The sensor tag 100 may be configured to be insertable into a container. The sensor tag 100 may comprise a capacitance sensor 115, a chip 110, and a radio-frequency identification (RFID) inlay 105. These components are indicated with the reference numerals 105 RFID, 110 Chip, indicating that the RFID and chip components are located at the top of the stick. Further down the stick, the label Capacitance Sensor 115, indicating the location of the cap sensor along the length of the stick. In one or more embodiments, the chip and RFID may be integrated in one component while the cap sensor is extended at a separate location of the sensor tag.

Referring to FIG. 1B, shows five sensor tag devices 100, each with distinct top sections containing RFID chips 105 and elongated bodies incorporating capacitance sensors 115. These configurations may demonstrate various arrangements of RFID chip placement 105 and capacitance sensor 115 integration. The figure shows five elongated sensor tags of the liquid level detection system arranged side by side in a row. Each sensor tag device 100 of a liquid level detection system 220 may be configured to be insertable into a container. The sensor tag 100 may comprise a capacitance sensor 115, a chip 110, and a radio-frequency identification (RFID) inlay 105 which is depicted in various shapes and internal configurations. In one or more embodiments, the chip and RFID may be integrated in one component while the cap sensor is extended at a separate location of the sensor tag.

Referring to FIGS. 1C and 1D, the liquid measurement system 220 shows a sensor tag 100 configuration may be shown with a branded section 117. The sensor tag 100 may comprise a liquid measurement strip 115 with a designated space 117 for branding of the beverage or product. The branded tag section 117 may display product identification information, including brand names, alcohol content percentages, or other product-specific markings. The RFID chip 105 may be positioned adjacent to the branded tag section 117 for optimal readability by external reader devices.

The branded tag section 117 may serve dual purposes of product identification and system functionality. The branding may include text such as “brand name” and “alcohol 40%” as shown in the figure, providing visual identification of the container contents. The capacitance sensors 115 may extend along the length of the liquid measurement strip below the branded section 117. This configuration may enable both liquid level measurement and product identification within a single integrated sensor tag system.

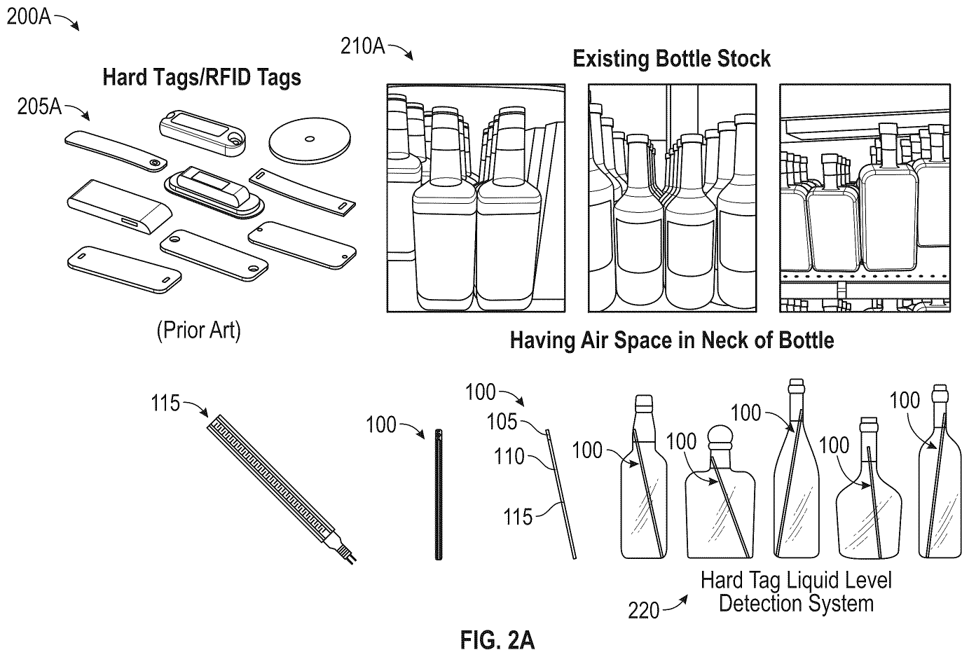

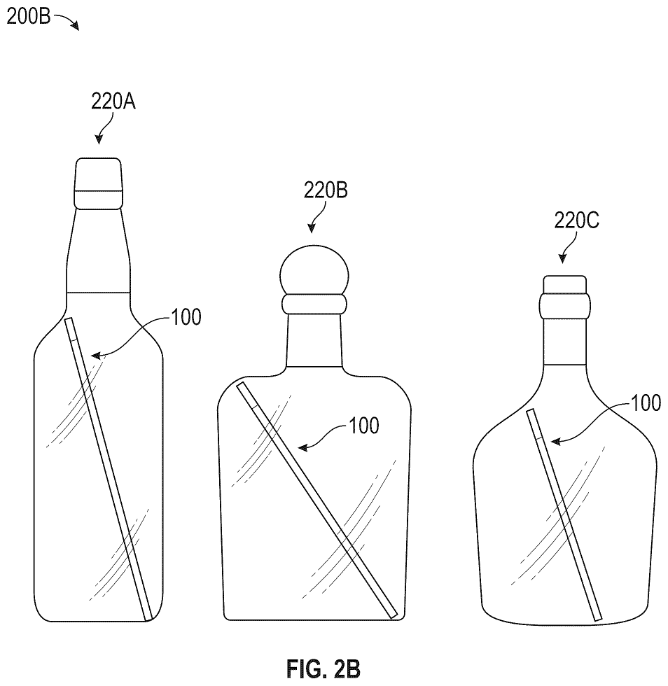

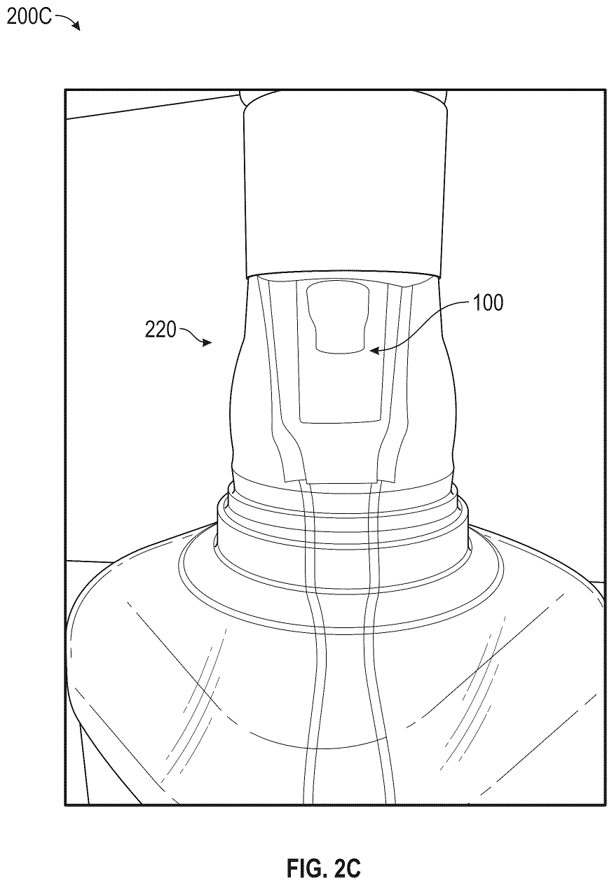

With reference to FIG. 2A, prior art components and existing bottle stock configurations may be illustrated alongside the hard tag liquid level detection system 220. The figure may show various hard tags and RFID tags 205A representing existing technologies in the field. The existing bottle stock 210A may demonstrate typical container configurations with air space in neck areas, which may present challenges for accurate liquid level detection using conventional methods.

The liquid level detection system 220 may address limitations of prior art approaches by integrating the sensor tag 100, bottle 210, and tracking capabilities into a unified system. The capacitance sensor 115 may provide accurate liquid level measurements even in containers with varying neck geometries. The system 220 may overcome challenges associated with air space variations in bottle necks by utilizing capacitance-based sensing technology that may function regardless of air gap configurations.

Referring to FIG. 2B, bottle configurations 200B may demonstrate sensor placement in various container geometries. The figure may show three different bottle shapes 220A, 220B, and 220C, each with sensor tag 100 placement configurations. top of each sensor tag device 100 may indicate different identification systems or sensor types corresponding to specific container configurations. In one or more embodiments, the sensor tag 100 placement may accommodate bottles with varying internal geometries while maintaining accurate liquid level detection capabilities. The sensor configurations may be optimized for different bottle shapes, including standard cylindrical bottles, tapered bottles, and specialty container designs. The color-coded identification system may enable rapid visual identification of sensor types or measurement ranges appropriate for specific bottle configurations.

With reference to FIG. 2C, a sensor tag 100 fully submerged configuration 200C may be depicted. The sensor tag may be shown completely surrounded by liquid within the container neck area. The liquid measurement sensor tag system 220 may maintain full operational capability even when the sensor tag may be entirely submerged in liquid. The encapsulation materials may provide complete protection against liquid exposure while preserving RFID communication functionality.

The fully submerged configuration may demonstrate the robust design of the sensor tag system. The sensor tag 100 may continue to provide accurate capacitance measurements and RFID communication capabilities even when completely immersed in various liquid types. The alcohol-resistant and chemical-resistant encapsulation materials may ensure long-term functionality in challenging liquid environments. The system may maintain readability through liquid media, enabling accurate inventory tracking regardless of liquid level height relative to the sensor tag position.

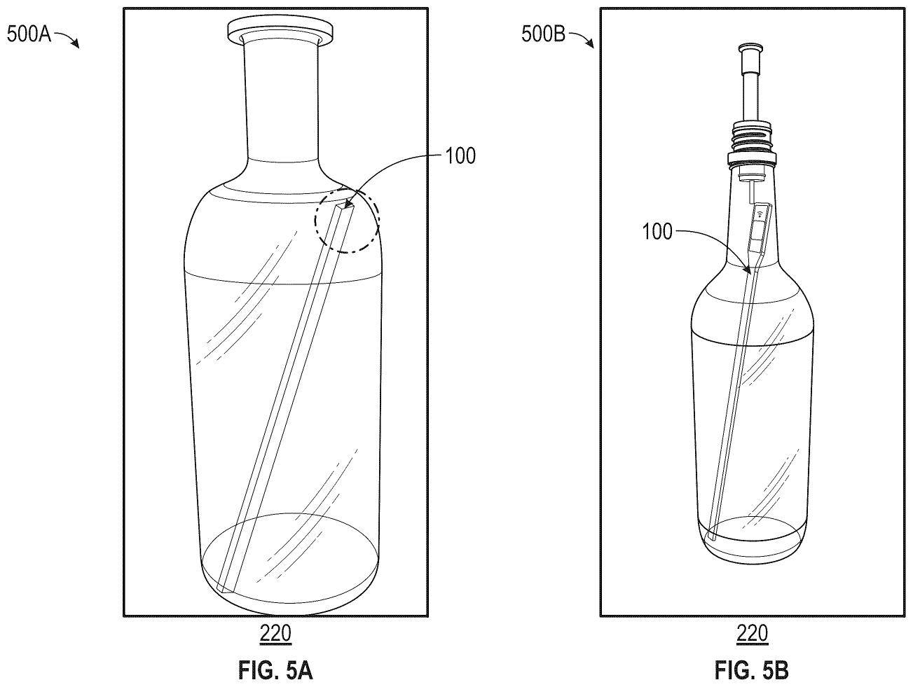

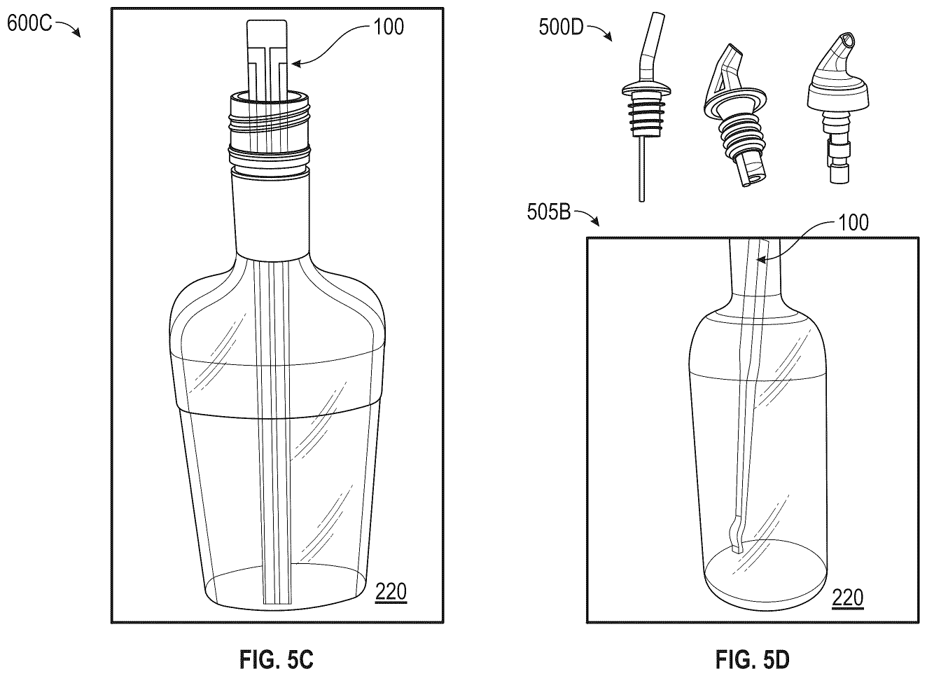

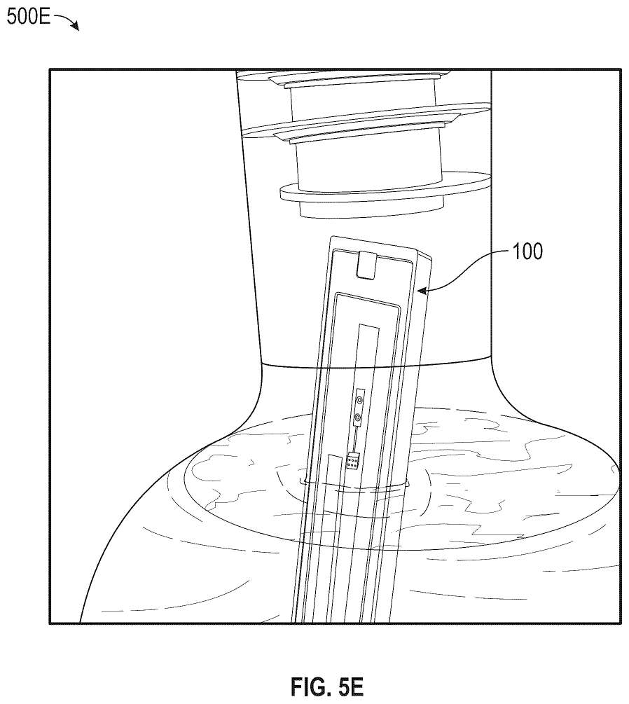

FIGS. 5A, 5B, 5C, 5D and 5E demonstrate various implementations of the liquid level detection system 220 with sensor tag 100 in different container configurations. The sensor tag 100 may be positioned within glass containers to extend vertically from bottle necks toward bottle interiors while maintaining contact with liquid contents. The sensor tag 100 may remain accessible for RFID communication and may accommodate different pour spout designs 500D. The sensor tag 100 may be configured to operate when partially or fully submerged in container contents while maintaining compatibility with existing bottle configurations and pouring capabilities.

### System of Liquid Measurement Tracking

The system of liquid measurement 220 utilizes the patented radio-frequency identification (RFID) capacitance liquid measurement tag system referenced in priority documents section the with the newly created sensor tag 100. The system 220 may operate through integration of multiple sensor tags 100 deployed across various containers within a monitoring environment. The sensor tags 100 may be configured to communicate wirelessly with handheld reader devices, fixed reader installations, and mobile applications to provide comprehensive liquid level tracking capabilities.

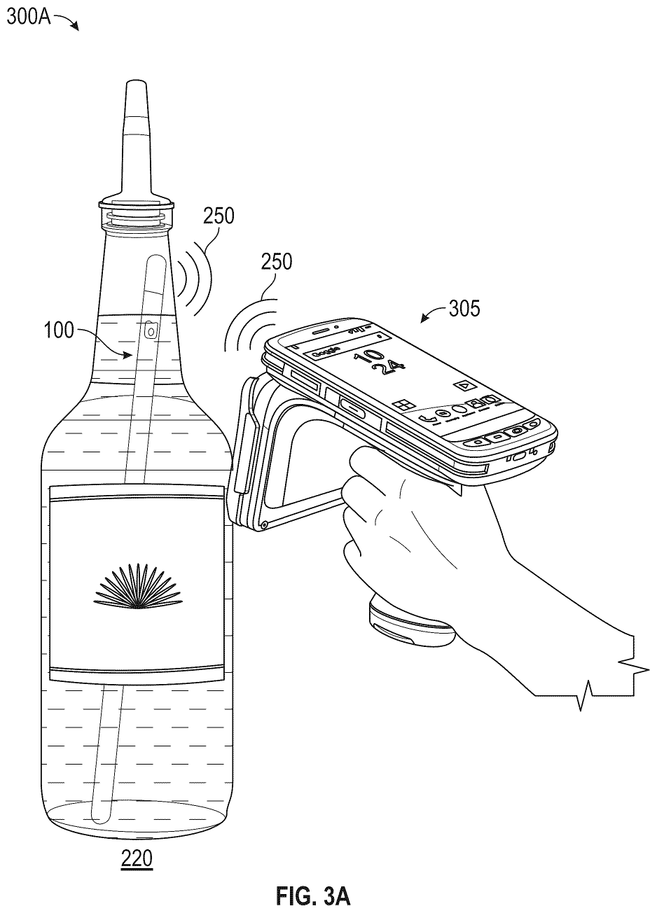

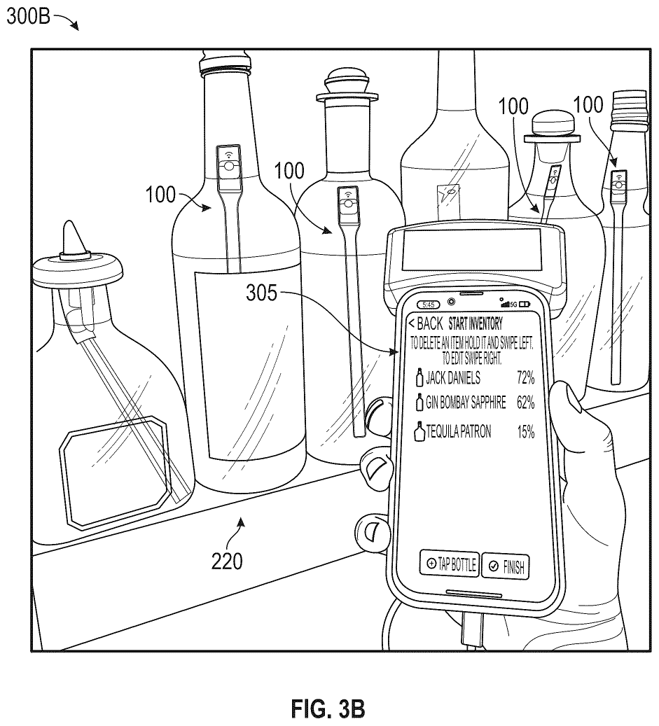

With reference to FIG. 3A, a handheld reader device 305 may scan an RFID capacitance liquid measurement sensor tag system 220 configured for liquid measurement applications. The handheld device 305 may be positioned adjacent to a container containing a sensor tag 100. Wireless communication signals 250 may be transmitted between the sensor tag 100 and the reader device 305. The reader device 305 may interrogate the sensor tag 100 to obtain liquid level measurements and associated data. The communication may occur through RFID protocols that enable the reader device 305 to receive capacitance-based liquid level information from the sensor tag 100.

Referring to FIG. 3B, multiple sensor tags 100 may be read by a handheld device with extended range capabilities for the RFID capacitance liquid measurement sensor tag system. The handheld device may display an inventory interface showing multiple beverage containers with their respective liquid level percentages. The device may simultaneously communicate with several sensor tags 100 positioned in different containers within the scanning range. The extended range capabilities may enable the reader device to collect data from multiple sensor tags without requiring individual proximity scanning of each container.

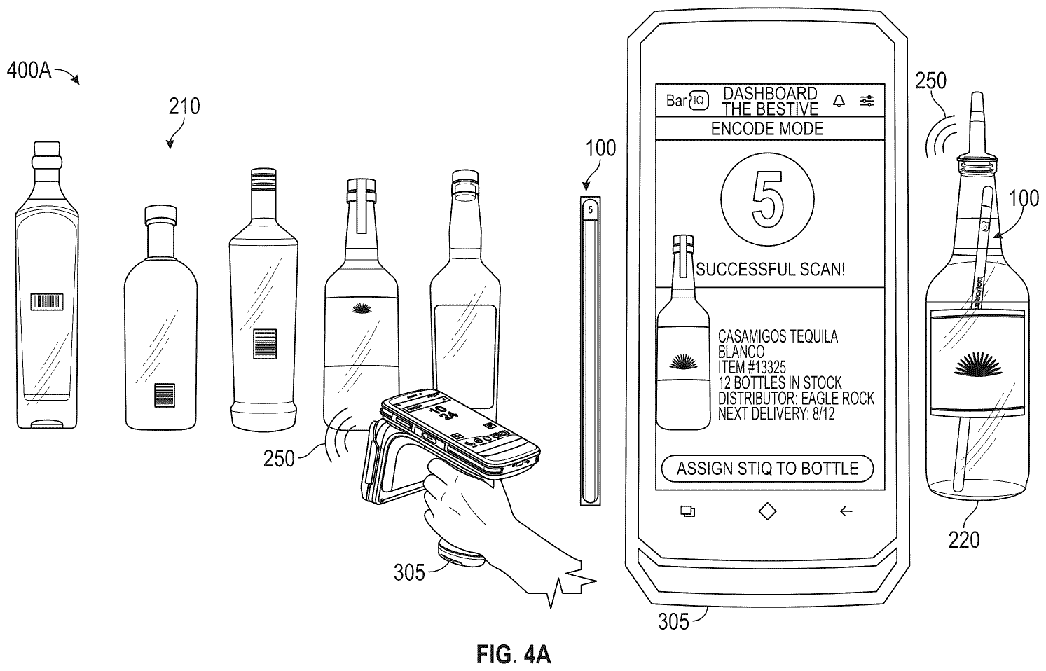

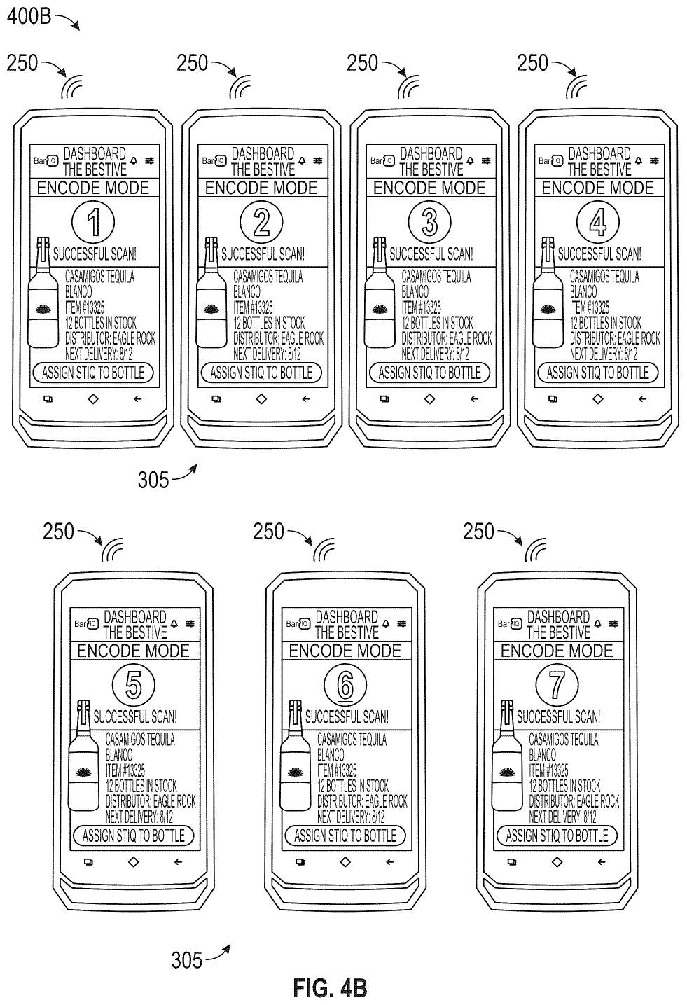

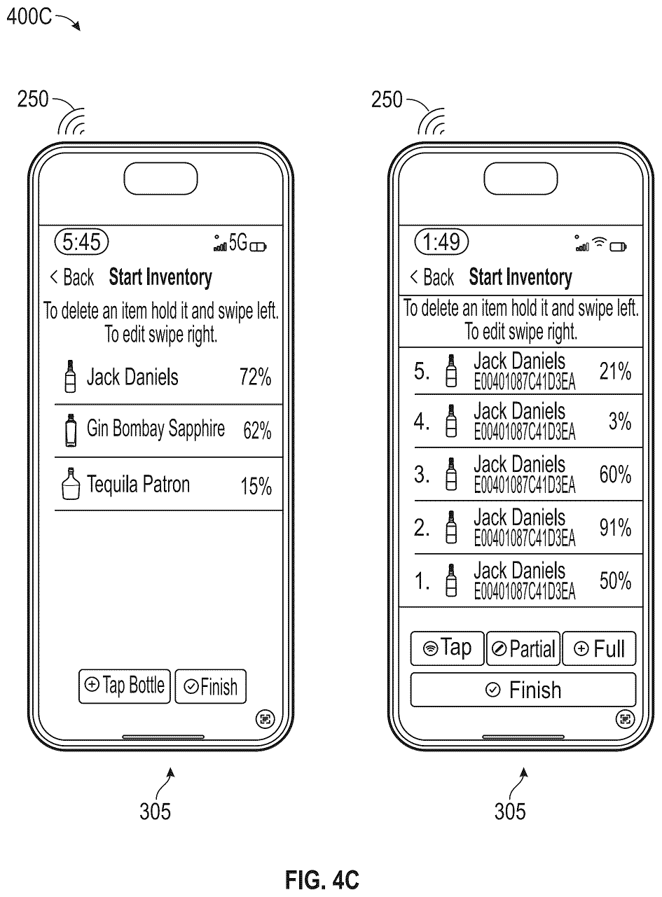

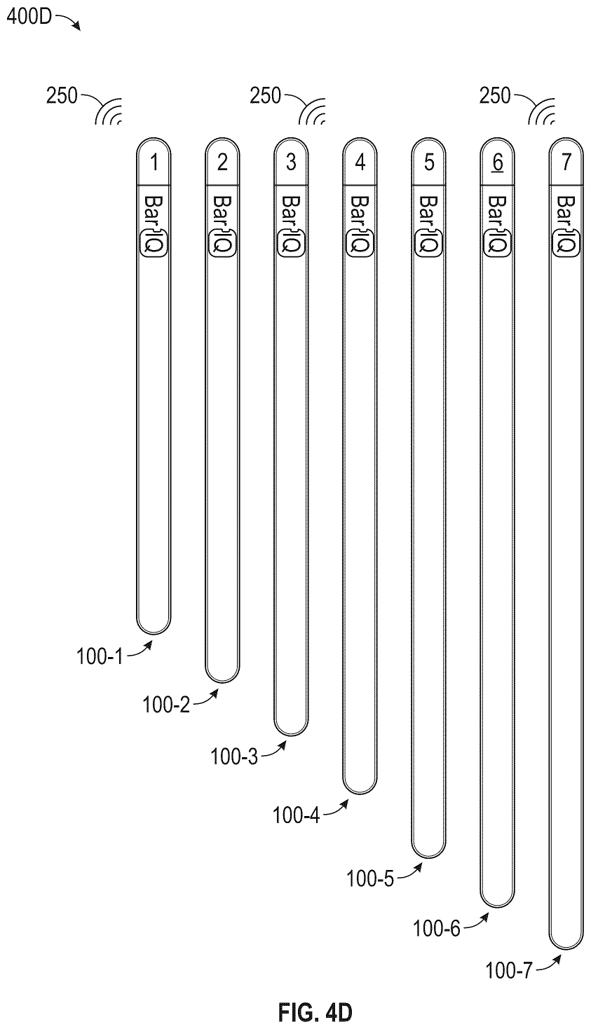

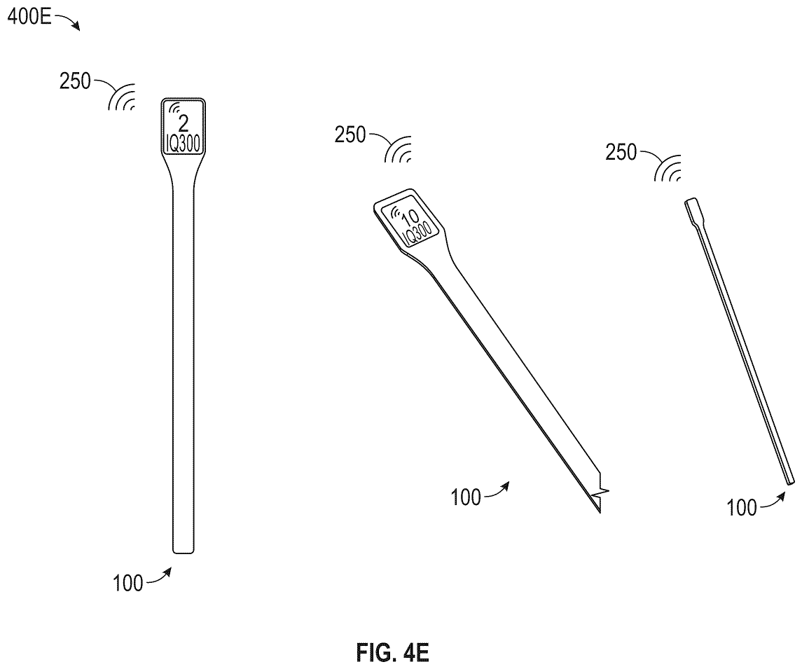

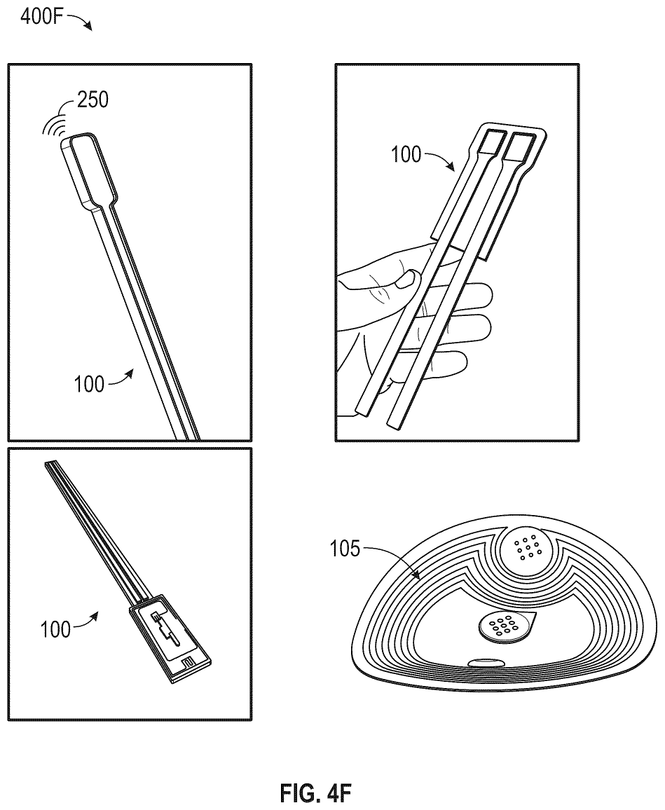

With reference to FIG. 4A, a process for determining sensor tag selection with scanned containers may be illustrated. The process may involve scanning containers 210 with a handheld reader device 305 to identify container characteristics. The system may analyze container dimensions, volume, and geometry to recommend appropriate sensor tag lengths and configurations. A mobile device interface may display encoding modes and successful pairing confirmations between sensor tags 100 and their assigned containers. The selection process may ensure optimal sensor tag placement for accurate liquid level detection across various container types.

Referring to FIG. 4B, mobile device interfaces may display color-coded identification systems for the RFID capacitance liquid measurement sensor tag system. Multiple smartphones may show identical interfaces with different colored backgrounds and numeric labels. Each interface may display sensor tag identification numbers and associated container information. The color-coding system may enable rapid visual identification of sensor tags and their corresponding containers during inventory management operations. The wireless communication capabilities 250 may be indicated for each mobile device interface.

With reference to FIG. 4C, color-coded and numbered liquid level detection sensor tags may be shown. Seven sensor tags 100-1 through 100-7 may be arranged with different colored identification markers numbered sequentially from 1 to 7. Each sensor tag may incorporate RFID components 105 and may be labeled with brand information, messages, logos, or other copy for easy identification. The color-coding and numbering system may facilitate quick selection of appropriate sensor tags for different container sizes and applications. The visual identification system may reduce installation errors and improve operational efficiency.

Referring to FIG. 4D, hard sensor tag units with identification features may be illustrated. The sensor tags may feature rectangular heads with numeric identifiers, wireless communication symbols, and alphanumeric codes. The identification features may include color-coded squares displaying numbers, text, and logos for easy identification and operational efficiency. The wireless communication capabilities may be indicated through signal icons 250 positioned above each sensor tag 100. The identification system may enable systematic organization and deployment of sensor tags across multiple containers.

With reference to FIGS. 4E and 4F, different types and views of RFID sensor tags may be displayed. The figure may show various sensor tag designs including elongated narrow configurations, handheld size comparisons, and curved RFID tag variants. The sensor tags 100 may be shown in both schematic and photographic representations to illustrate different form factors and applications. The RFID components 105 may be visible in some configurations, demonstrating the integration of wireless communication capabilities within the sensor tag designs.

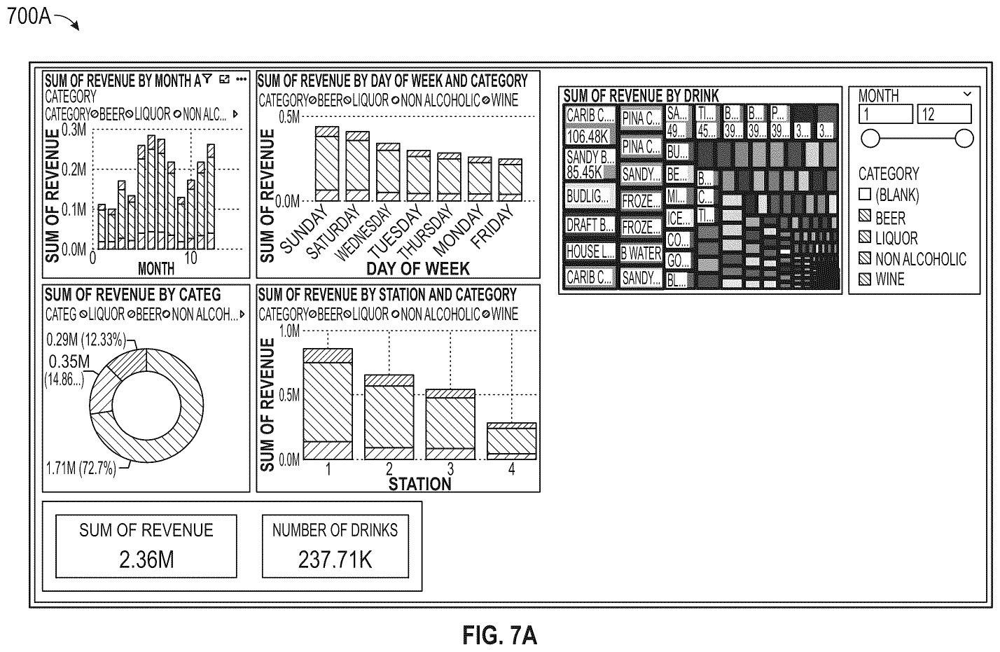

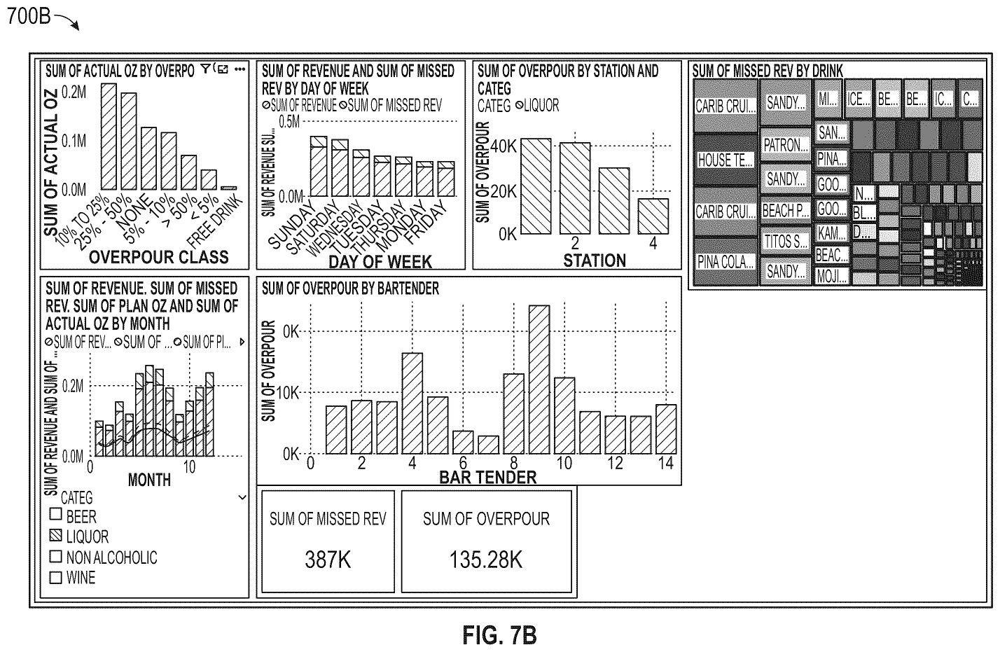

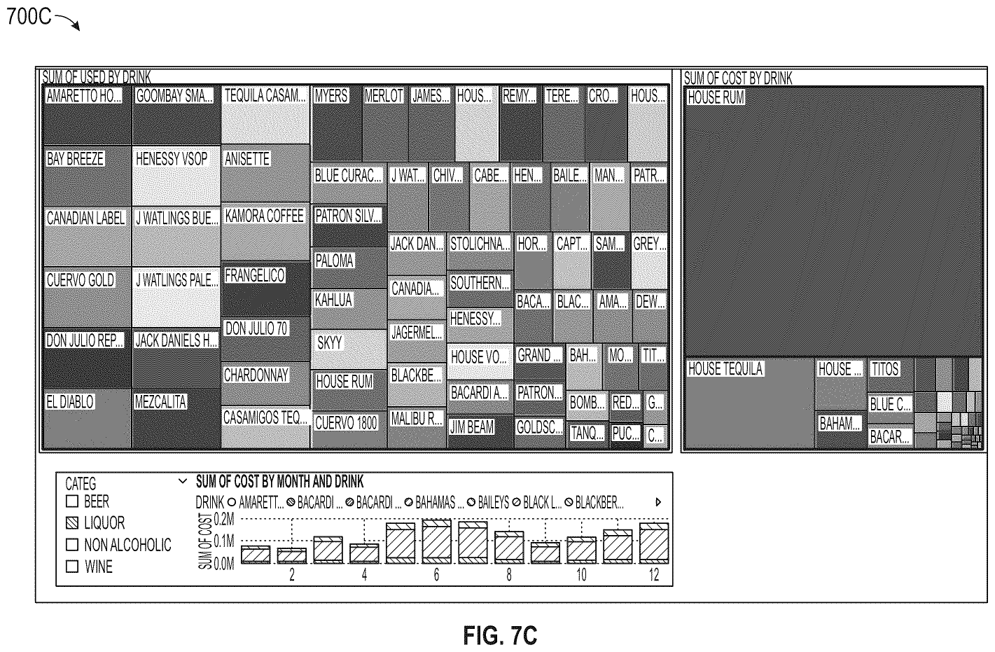

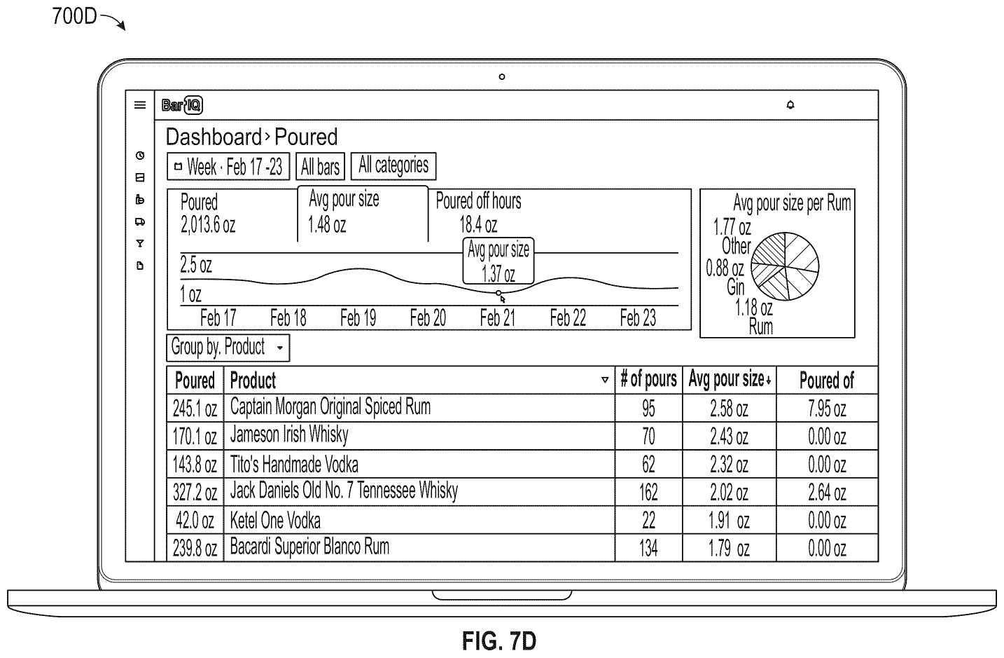

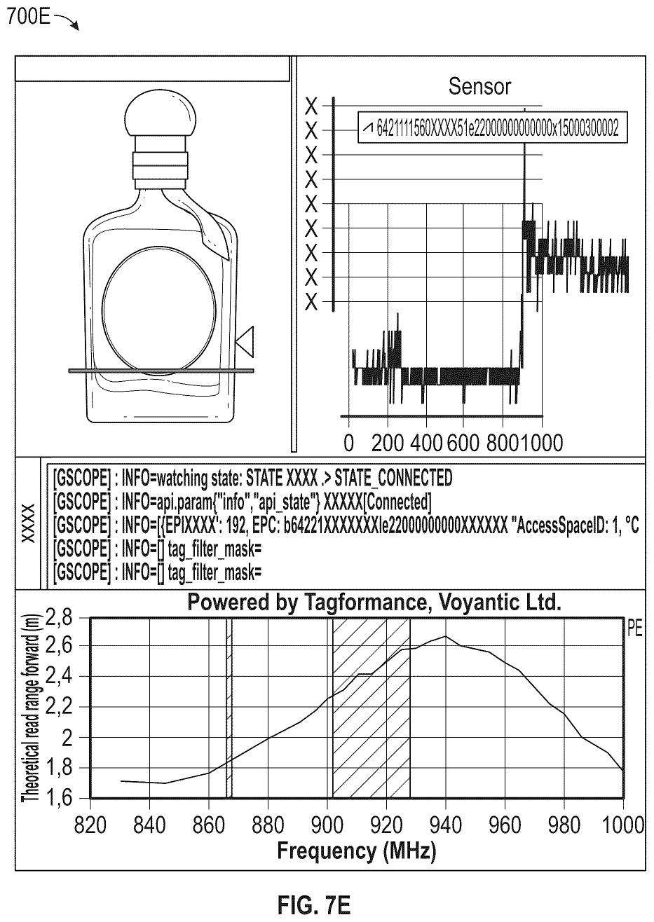

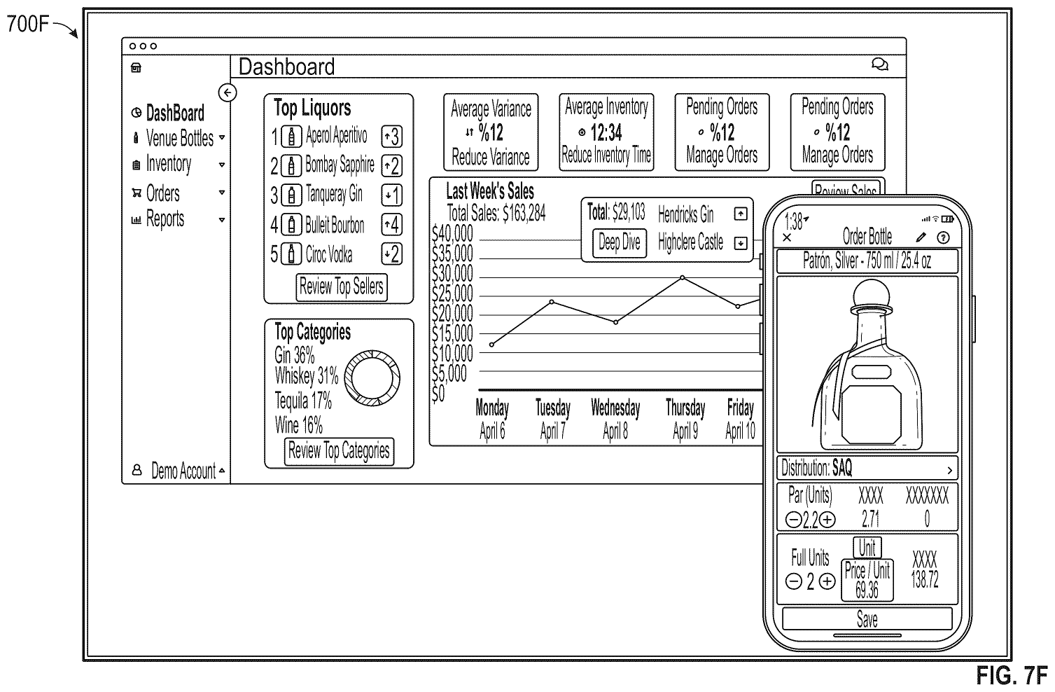

Referring to FIG. 7A, a user platform dashboard interface configuration may be shown. The dashboard 700A may display multiple data visualization panels including bar charts for revenue analysis, charts for drink categorization, and summary statistics. The interface may process data collected from multiple sensor tags 100 to generate comprehensive inventory and sales analytics. The dashboard may provide real-time monitoring capabilities for beverage inventory levels, consumption patterns, and financial performance metrics derived from sensor tag measurements.

With reference to FIG. 7B, a dashboard interface with analytics and reporting features may be illustrated. The interface 700B may include charts for overpour analysis, revenue tracking, and bartender performance monitoring. The system may calculate missed revenue and overpour amounts based on liquid level changes detected by the sensor tags 100. The analytics may provide insights into operational efficiency and inventory management performance through automated data collection from the RFID capacitance liquid measurement sensor tag system.

Referring to FIG. 7C, a dashboard interface with analytics and reporting features may be illustrated. The visualization 700C may show drink usage and cost data through colored rectangles representing different beverages alongside bar charts for monthly analysis. The map-style visualization may display relative usage volumes for different liquor types based on liquid level measurements from sensor tags 100. The cost analysis may be derived from consumption data automatically collected through the RFID sensor tag system.

With reference to FIG. 7D, a laptop computer may display dashboard interface 700D. The application may show metrics for poured amounts, average pour sizes, and trend analysis through line graphs and pie charts. The dashboard may process data from multiple sensor tags 100 to calculate precise pour measurements and consumption patterns. The interface may provide real-time monitoring of beverage inventory levels and usage statistics for operational decision-making.

Referring to FIG. 7E, sensor data visualization with graphs and terminal output may be illustrated. The FIG. 700E may show a bottle image with sensor data, frequency analysis graphs, and technical performance metrics. The sensor tag 100 may provide capacitance measurements that can be processed and displayed as graphical data representations. The technical output may include RFID communication status, sensor performance parameters, and measurement accuracy indicators for system monitoring and troubleshooting.

With reference to FIG. 7F, integrated dashboard and mobile ordering interface 700F may be shown. A computer monitor may display management dashboard panels while a smartphone may show product ordering interfaces for beverage inventory. The system integration may enable automated reordering based on liquid level thresholds detected by sensor tags 100. The mobile interface may provide field personnel with access to inventory data and ordering capabilities derived from real-time sensor measurements.

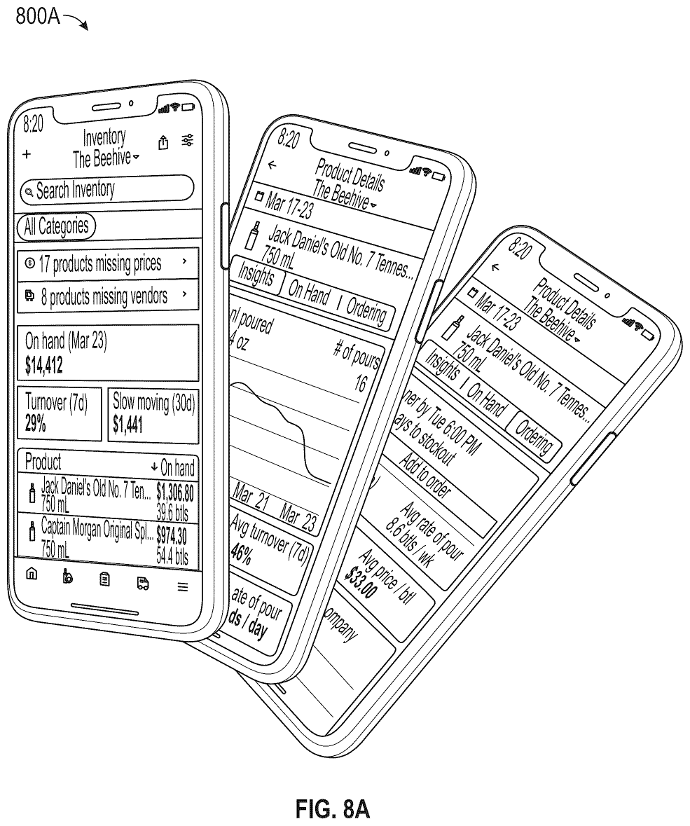

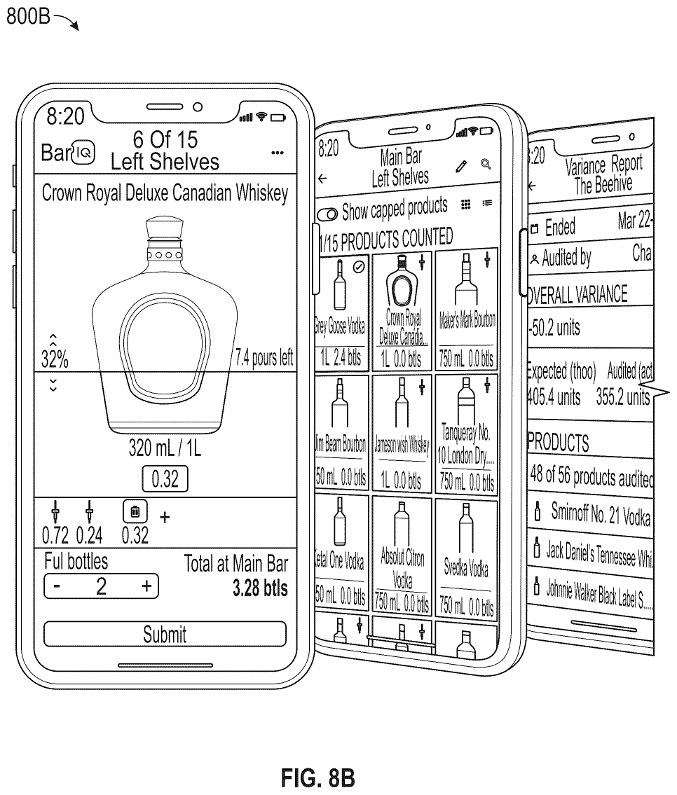

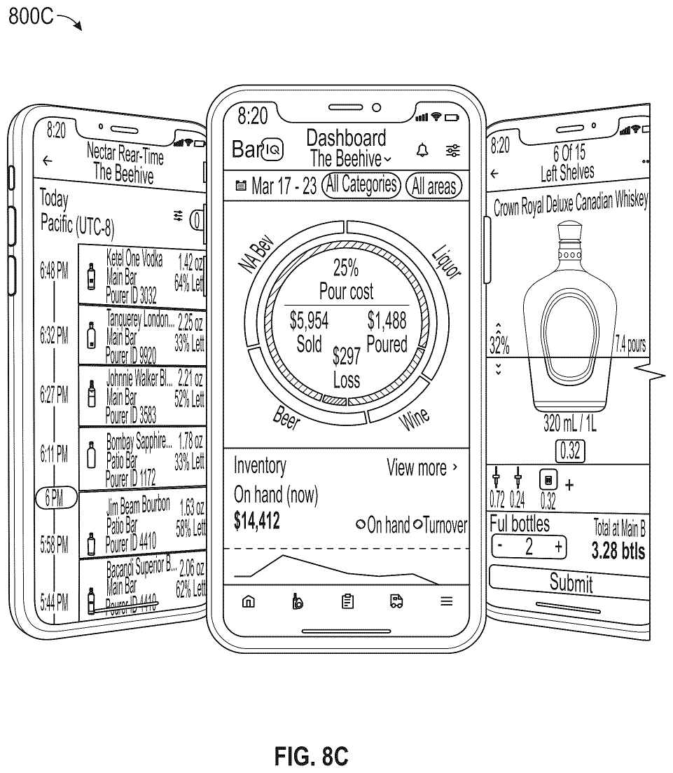

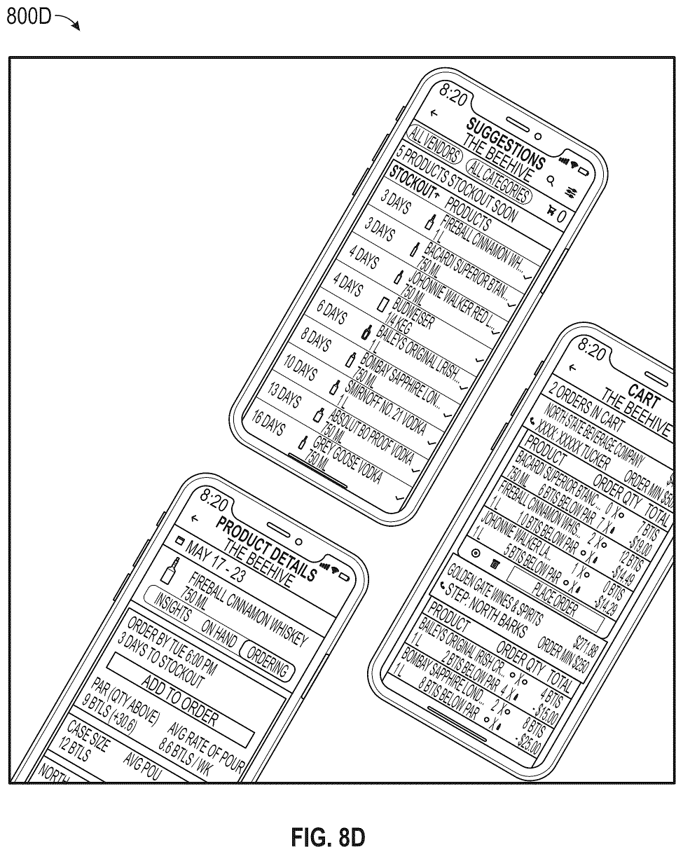

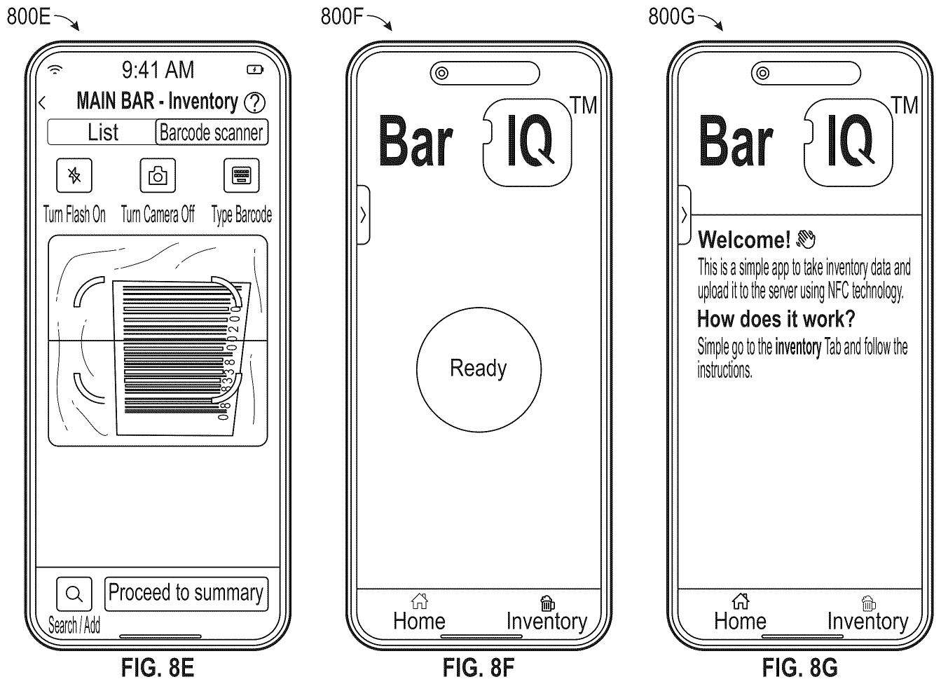

Referring to FIG. 8A, mobile application screens 800A may provide inventory management functionality. Three smartphones may display different screens including inventory dashboards, product lists, sales graphs, and detailed product information. The mobile interfaces may process data from sensor tags 100 to provide real-time inventory status, reorder recommendations, and consumption analytics. The application may enable field inventory management through wireless communication with deployed sensor tags.

With reference to FIG. 8B, mobile interface screens 800B may show product details and variance reporting. The smartphones may display detailed product pages with liquid level measurements, shelf overviews with bottle images, and variance reports comparing expected versus audited values. The variance analysis may be based on automated measurements from sensor tags 100 compared to manual inventory counts or expected consumption patterns.

Referring to FIG. 8C, mobile dashboard interfaces 800C may provide inventory analytics. The screens may show spirit inventories, dashboard analytics with pour cost percentages, and detailed product information for inventory management. The mobile interface may display real-time liquid level data collected from sensor tags 100 to provide immediate inventory status and operational metrics for beverage management applications.

With reference to FIG. 8D, mobile shopping and ordering interfaces 800D may be illustrated. The smartphones may display beverage selection lists, detailed product pages with pricing, and shopping cart summaries with checkout options. The ordering system may be integrated with inventory data from sensor tags 100 to provide accurate stock availability and automated reordering capabilities based on current liquid levels.

Referring to FIG. 8E, a barcode scanning interface 800E may be shown for inventory applications. The mobile interface may display barcode scanner functionality with flash controls and search capabilities for inventory management. The barcode scanning may be used in conjunction with sensor tag 100 data to provide comprehensive inventory tracking that combines automated liquid level measurement with product identification capabilities.

With reference to FIG. 8F, a mobile application home screen 800F may be illustrated. The “BarIQ™” application may show a simple interface with a central “Ready” button and navigation icons for Home and Inventory functions. The application may serve as the primary interface for interacting with the RFID capacitance liquid measurement sensor tag system, providing access to sensor data and inventory management features.

Referring to FIG. 8G, a welcome screen 800G may provide NFC technology instructions. The “Bar IQ™” application may display welcome messages explaining NFC-based inventory data collection and upload functionality. The interface may provide user guidance for operating the RFID capacitance liquid measurement sensor tag system through Near Field Communication protocols for data collection and server synchronization.

### Methods of Packaging and Delivery

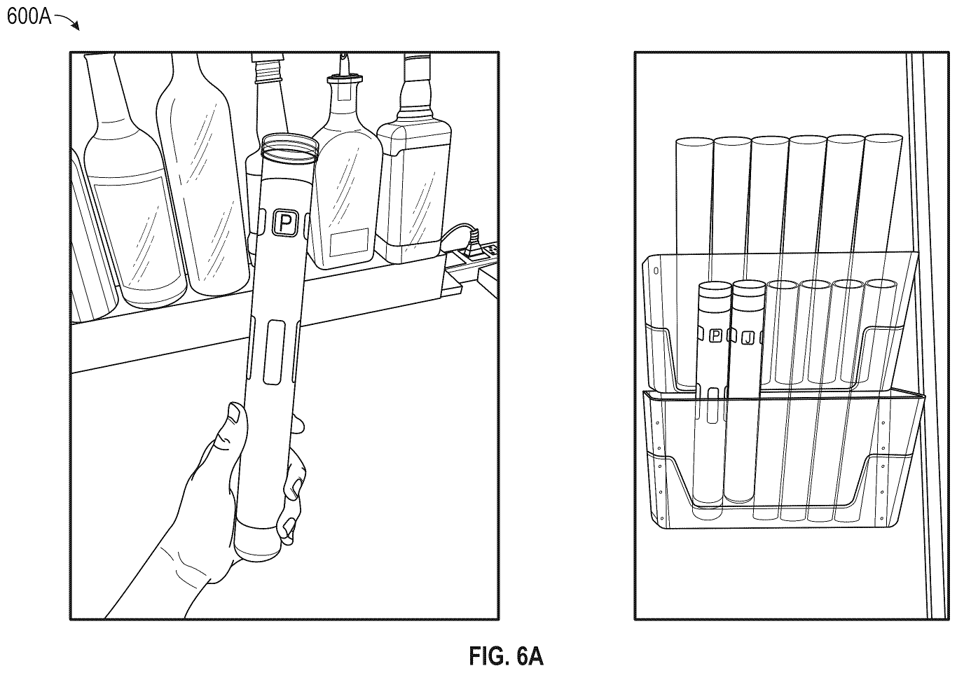

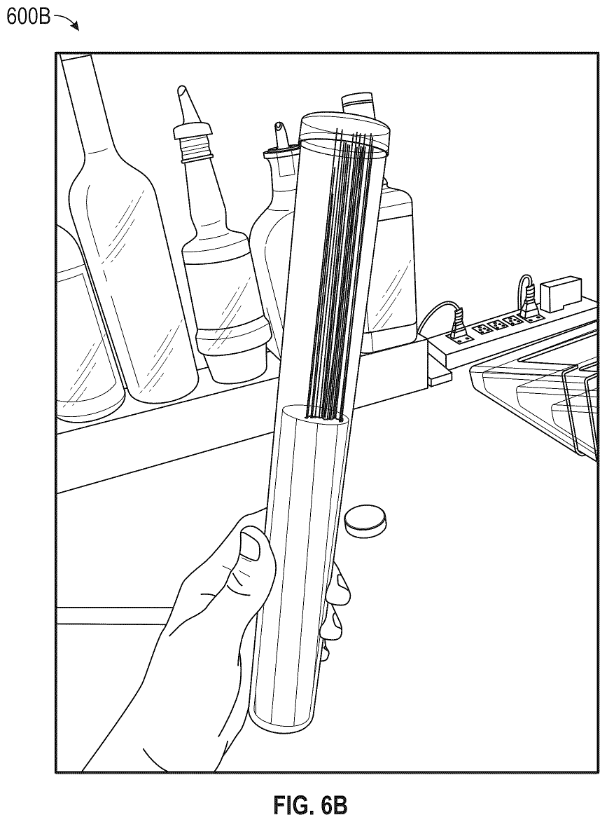

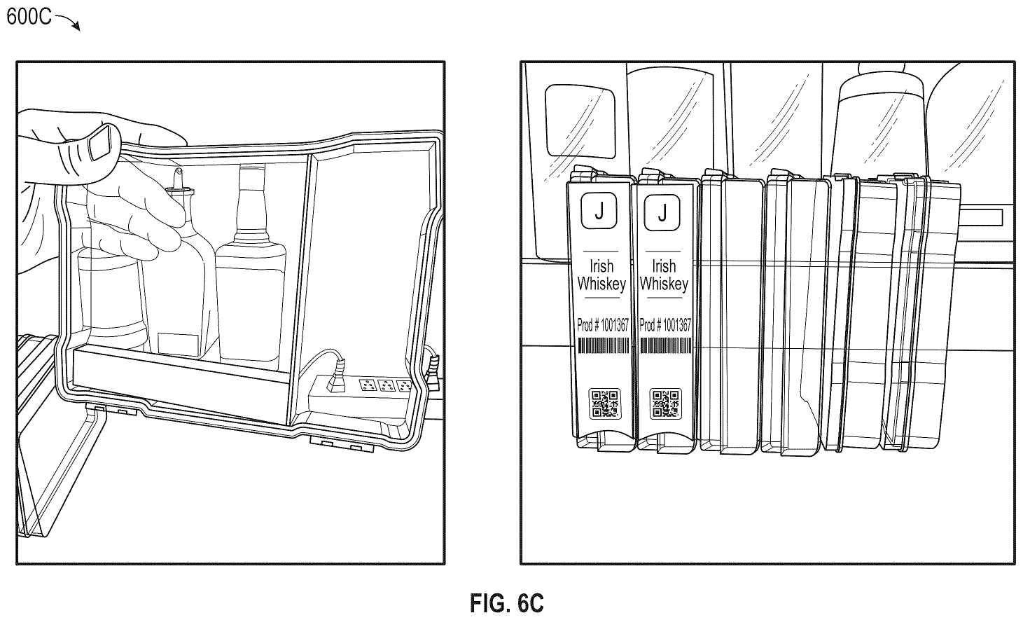

FIG. 6A shows a packaging delivery system for sensor tags. The system may comprise tubular containers configured to store and dispense sensor tags in an organized manner. The tubular containers may be arranged alphabetically and may be positioned in linear configurations without requiring additional shelving or storage hardware. The containers may function as a modular system that may be self-contained.

The tubular containers may be arranged in multiple orientations. The containers may be positioned vertically to optimize space utilization. The containers may include clear markings, color coding, and identification information to enable rapid sensor tag selection. A QR code may be displayed on the container graphics to facilitate product reordering through scanning functionality.

Transparent sections of the tubular containers may allow visual inspection of sensor tag inventory levels. The transparent areas may serve as visual indicators of remaining sensor tag quantities without requiring container opening. A hinged cap or flip cap may be positioned at the top portion of each tubular container. The cap mechanism may enable single-handed operation for container opening, sensor tag retrieval, and container closure.

A magnetic element may be positioned at the bottom portion of each tubular container. The magnetic element may secure the containers to metallic surfaces commonly found in inventory rooms, offices, kitchen areas, and industrial environments. The magnetic attachment may maintain container stability during use.

The tubular containers may accommodate multiple sensor tag lengths through standardized container dimensions. Foam spacers may be inserted within the containers to accommodate different sensor tag lengths. The foam spacers may enable the use of uniform container sizes regardless of sensor tag dimensions.

FIG. 6A may depict a packaging delivery system 600A for sensor tags. The tubular containers may be arranged in a row configuration to facilitate efficient sensor tag storage and dispensing. The containers may include transparent sections that allow visual inspection of sensor tag inventory levels without requiring container opening. Each tubular container may feature a hinged cap mechanism positioned at the top portion to enable single-handed operation during sensor tag retrieval.

FIG. 6B may illustrate the magnetic attachment feature of the tubular containers 600B. The magnetic element may be positioned at the bottom portion of each container to secure the containers to metallic surfaces commonly found in inventory storage areas. The magnetic attachment system may provide stability during container use while maintaining the modular arrangement of the packaging delivery system. The containers may accommodate different sensor tag lengths through the use of foam spacers that may be inserted within the containers to maintain uniform container dimensions regardless of sensor tag size variations. Therefore, FIG. 6B shows an alternative cartridge-based delivery system for sensor tags. The cartridge may incorporate gravity-fed or spring-loaded dispensing mechanisms. The dispensing mechanisms may facilitate sensor tag retrieval. The cartridge may include a dispensing slot configured to release individual sensor tags. The slot mechanism may enable controlled sensor tag dispensing.

FIG. 6C depicts a view 600C which may illustrate an alternative packaging configuration for cartridge-based delivery of the sensor tags 100. The cartridge system may provide organized storage and dispensing mechanisms for multiple sensor tags 100. The packaging approach may enable efficient distribution and deployment of sensor tags 100 across various container applications.

### Agricultural Applications

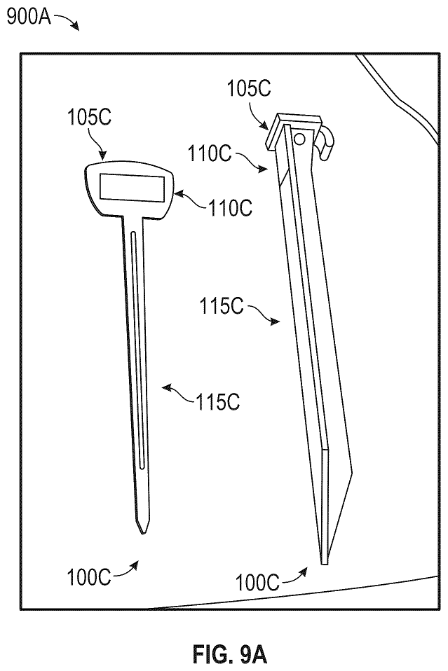

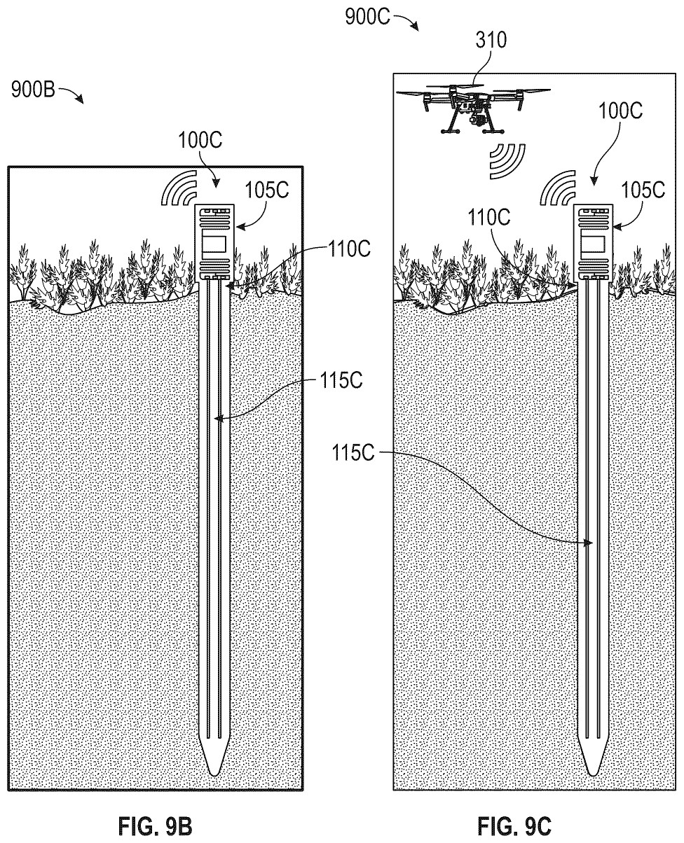

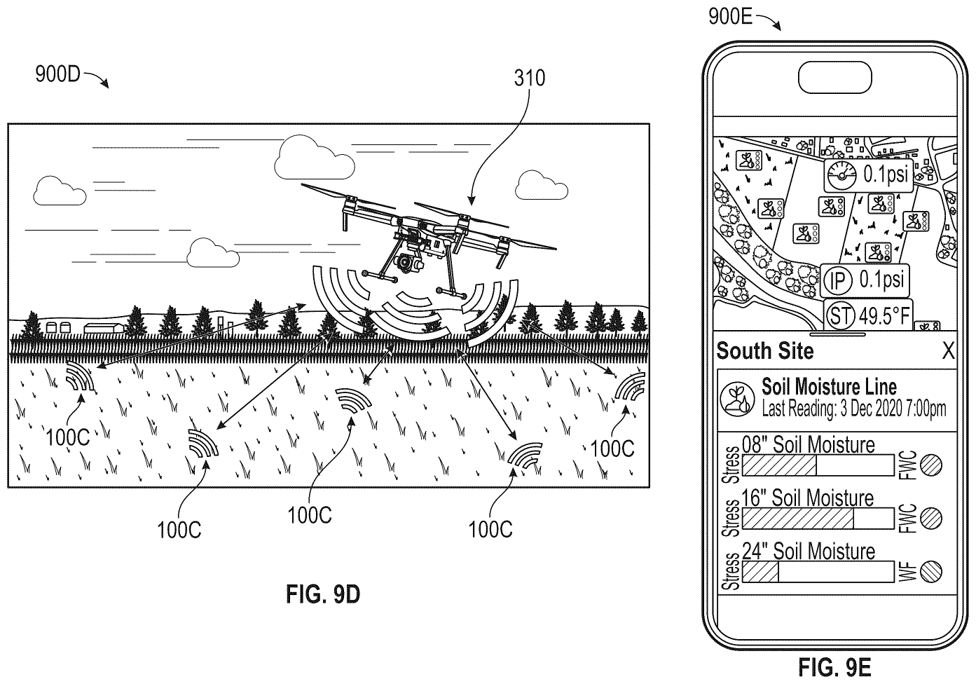

In agricultural soil environments, the liquid measurement system 220 may be configured to provide comprehensive soil monitoring capabilities through specialized sensor deployment and data collection methods. As shown in FIG. 9A, 900A, the sensor tag 100C may be adapted for insertion into agricultural soil to depths of six inches or more below the surface. The system may monitor volumetric water content, soil water tension, and temperature conditions at various depths within the growing medium.

In one or more embodiments, the agricultural implementation of the liquid measurement system 220 may utilize the same core capacitance sensing technology adapted for soil conditions rather than liquid containers. As shown in FIG. 9B, 900B, The RFID chip 105C The capacitance sensor 115C may detect changes in dielectric properties of soil as moisture content varies throughout different soil layers. The system may enable battery-free operation through passive wireless communication protocols including RFID, NFC, BLE, and 5G technologies for data transmission to external reader devices.

In one or more embodiments, the liquid level detection system may comprise a sensor tag 100C, wherein the sensor comprises a chip 110C, a capacitance sensor 115C, and a HarvestComm Unit (HCU) 105C. A HarvestComm Unit (HCU) is configured to harvest wireless energy and communicate over one or more of RFID, BLE (Bluetooth low energy), ambient IoT, Wi-Fi, 5G, mesh networking, zigbee, z-wave, NFC, cellular, ultra-wideband, ANT, and other capable emerging technology protocols. In one or more embodiments, the liquid level detection system may include a processing component 110C comprising at least one of a microcontroller, application-specific integrated circuit (ASIC), system-on-chip (SoC), field programmable gate array (FPGA), or digital signal processor (DSP). This may be embodied as a sensecore processor, a datafusion engine, a nanologic unit, a low-power ARM cortex-M chip, RISC-V core, embedded microchip, edgecompute node, embedded AI accelerator, TinyML chip, neuromorphic process or the likeness thereof.

The agricultural sensor tag 100C may be designed for long-term deployment, potentially lasting 10-20 years without maintenance requirements. The sensor may be inserted into soil using stake-like configurations through manual insertion methods or automated deployment systems. The system may support various reading methods including handheld UHF RFID readers, NFC-enabled smartphones, vehicle-mounted readers on tractors or ATVs, and drone-based data collection systems for comprehensive field coverage.

In one or more embodiments, the agricultural monitoring system may provide benefits including reduced irrigation costs, improved crop yields, prevention of over-watering and under-watering conditions, and promotion of sustainable farming practices. The system may enable data-driven irrigation decisions based on actual soil conditions rather than estimates or surface-level observations.

As shown in FIG. 9C, 900C and FIG. 9D, 900D, The wireless communication capabilities may enable large-area monitoring through drone-based 310 or vehicle-mounted reading systems, providing comprehensive field coverage for precision agriculture applications. The agricultural monitoring system may integrate with drone-based data collection platforms to enable comprehensive field coverage without requiring ground-based personnel to access each sensor location individually. The drone systems may be equipped with RFID reader antennas that may communicate with multiple sensor tags 100 during single flight operations. The aerial data collection may provide real-time soil moisture mapping across entire agricultural fields, enabling precision irrigation management and resource optimization.

In one or more embodiments, the agricultural embodiment may measure various soil parameters including volumetric water content, soil water tension or matric potential, soil temperature, and potentially pH levels. The system may provide real-time data collection capabilities when integrated with fixed reading stations or battery-enhanced configurations for signal amplification. The sensor deployment may be optimized for different crop types and field conditions through various sensor tag lengths and configurations.

In one or more embodiments, the system may support integration with existing agricultural equipment and irrigation systems through software interfaces and data processing components. The agricultural application may address critical needs in modern farming including water conservation, resource optimization, labor reduction through automated monitoring, and improved crop management through data-driven decisions. The system may contribute to sustainable agricultural practices by enabling precise resource application based on actual soil conditions rather than generalized irrigation schedules.

In one or more embodiments, the agricultural implementation may utilize specialized sensor deployment systems to facilitate efficient field installation and data collection across large farming operations. The liquid measurement system 220 may be adapted for soil insertion using automated deployment mechanisms that may enable rapid sensor placement across multiple field locations. The sensor tags 100C may be configured with reinforced housing materials designed to withstand soil penetration forces and long-term underground exposure conditions.

As shown in FIG. 9E, 900E, The agricultural sensor deployment may incorporate stake-like configurations with pointed insertion ends to facilitate penetration into various soil types and conditions. The sensor tags 100C-2 and 100C-3 may be manufactured in different lengths to accommodate varying soil depth requirements and crop-specific monitoring needs. The agricultural sensors may be designed to measure soil parameters at depths ranging from six inches to several feet below the surface, depending on crop root zone requirements and irrigation system configurations.

In one or more embodiments, the sensor tags 100C may be configured to operate in battery-free passive modes to eliminate maintenance requirements associated with power source replacement in field conditions. The passive operation may be enabled through inductive power harvesting from reader devices during data collection operations. The system may alternatively incorporate long-life battery configurations for applications requiring continuous monitoring or data logging capabilities between reader interrogation cycles.

In one or more embodiments, the agricultural liquid measurement system 220 may provide soil moisture data that may be processed to generate irrigation recommendations based on crop-specific water requirements and soil conditions. The system may integrate with existing farm management software platforms to provide automated irrigation scheduling and water application optimization. The data collection may enable farmers to implement precision agriculture practices that may reduce water consumption while maintaining or improving crop yields.

In one or more embodiments, the sensor deployment may be configured for various agricultural applications including row crops, orchards, vineyards, and greenhouse operations. The sensor tags 100 may be positioned at strategic locations throughout fields to provide representative soil moisture data for different soil types, topographical conditions, and irrigation zones. The system may support integration with existing irrigation infrastructure including drip systems, sprinkler systems, and center pivot irrigation equipment.

In one or more embodiments, the agricultural embodiment may incorporate weather-resistant encapsulation materials that may protect sensor components from moisture, temperature fluctuations, and chemical exposure from fertilizers and pesticides. The encapsulation materials may be selected to provide long-term durability in soil environments while maintaining accurate sensor performance throughout extended deployment periods. The sensor tags 100 may be designed to remain functional for multiple growing seasons without requiring replacement or maintenance.

As illustrated in FIG. 9F, 900F, the data processing components of the agricultural liquid measurement system 220 may provide analytics capabilities that may identify irrigation efficiency opportunities and water conservation potential. The system may generate reports comparing actual soil moisture conditions with optimal ranges for specific crops and growth stages. The analytics may enable farmers to implement data-driven irrigation decisions that may improve crop quality while reducing water usage and operational costs. The agricultural monitoring system may support integration with weather monitoring stations and meteorological data sources to provide comprehensive environmental monitoring capabilities. The combined soil moisture and weather data may enable predictive irrigation scheduling based on anticipated rainfall, temperature conditions, and evapotranspiration rates. The integrated approach may optimize irrigation timing and water application rates to maximize crop productivity while minimizing resource consumption.

### CONCLUSION

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and quantum computing elements. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

All rights including copyrights in the code included herein are vested in and the property of the Applicant. The Applicant retains and reserves all rights in the code included herein, and grants permission to reproduce the material only in connection with reproduction of the granted patent and for no other purpose.

All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose. Regarding the terms “BarIQ”, “LiquorStiq,” “Liquor Stick,” “Liquor Stiq” and the like, Applicants retain and reserve all rights in their trademarks and copyrights included herein, and grant permission to reproduce the material only in connection with reproduction of the granted patent and for no other purpose.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

Although very narrow claims are presented herein, it should be recognized the scope of this disclosure is much broader than presented by the claims. It is intended that broader claims will be submitted in an application that claims the benefit of priority from this application.

What is claimed is:

1. A liquid level detection system comprising:
a sensor tag insertable into a container, wherein the sensor tag comprises:
a capacitance sensor;
a chip;
a HarvestComm Unit (HCU);
wherein the capacitance sensor, the chip, and the HCU are encapsulated within the sensor tag; and
wherein the HarvestComm Unit is configured to harvest wireless energy and communicate utilizing at least one of the following protocols: RFID, Bluetooth low energy, ambient IoT, Wi-Fi, 5G, mesh networking, zigbee, z-wave, NFC, cellular, ultra-wideband, ANT, and other capable protocols.

2. The liquid level detection system of claim 1, wherein the sensor tag is fabricated from at least one of: a rigid material, a semi-rigid material, and a flexible material.

3. The liquid level detection system of claim 1, wherein the sensor tag is embodied as a rigid sensor tag, a hard sensor tag, a rod, a shaped object, or a stick, and wherein the encapsulation material comprises at least one of glass, foam, rubber, silicone, ceramic, PVC, wood or natural fiber, synthetic fiber, metal, alcohol-resistant plastic, or BPA-free plastic.

4. The liquid level detection system of claim 1, wherein the sensor tag is encapsulated with a material comprising at least one of: BPA-free plastic, laminated sheets, and other encapsulating materials.

5. The liquid level detection system of claim 1, wherein the sensor tag is provided in a plurality of lengths.

6. The liquid level detection system of claim 5, wherein each length of the plurality of lengths is associated with a unique identifier.

7. The liquid level detection system of claim 6, wherein the unique identifier comprises at least one of a color, a number, a symbol, a logo mark, or brand information.

8. The liquid level detection system of claim 1, wherein the sensor tag further comprises a weighting element at a bottom portion of the sensor tag.

9. The liquid level detection system of claim 1, wherein the HCU is positioned near a top portion of the sensor tag.

10. The liquid level detection system of claim 1, wherein the sensor tag comprises a battery-free inlay configured to operate without an internal battery.

11. The liquid level detection system of claim 1, wherein the sensor tag further comprises an internal battery encapsulated within the sensor tag.

12. The liquid level detection system of claim 1, wherein the capacitance sensor is configured to detect changes in capacitance as a liquid level in the container varies.

13. The liquid level detection system of claim 12, wherein the chip is configured to process the changes in capacitance detected by the capacitance sensor.

14. The liquid level detection system of claim 13, wherein the HCU is configured to transmit data processed by the chip to an external reader device.

15. A liquid inventory management system comprising:
a plurality of sensor tags, each sensor tag insertable into a container and comprising a capacitance sensor, a chip, and a radio-frequency identification (RFID) inlay encapsulated within the sensor tag;
at least one RFID reader device configured to receive data from the plurality of sensor tags; and
a data processing component configured to analyze the received data and generate inventory status reports.

16. A method for liquid level detection using the system of claim 15, the method comprising:
inserting one of the plurality of sensor tags into a container;
detecting, by the capacitance sensor of the sensor tag, changes in capacitance as a liquid level in the container varies;
processing, by the chip of the sensor tag, the detected changes in capacitance;
transmitting, by the RFID inlay of the sensor tag, the processed data to the at least one RFID reader device; and
analyzing, by the data processing component of the system, the transmitted data to generate an inventory status report.

17. A fluid and soil monitoring system comprising:
a plurality of sensor devices, each sensor device insertable into a medium selected from a liquid container or soil, wherein each sensor device comprises:
a capacitance sensor configured to detect a level condition of the medium, the level condition comprising at least one of a liquid level or a soil moisture level;
a processing chip configured to process capacitance data from the capacitance sensor; and
a wireless communication module configured to transmit the processed capacitance data to an external reader device;
wherein the capacitance sensor, the processing chip, and the wireless communication module are encapsulated within a rigid housing configured for penetration into the medium.

18. The fluid and soil monitoring system of claim 17, wherein the rigid housing is configured for soil penetration to a depth of at least six inches below a surface.

19. The fluid and soil monitoring system of claim 17, wherein the wireless communication module comprises at least one of RFID, NFC, BLE, or 5G technology.

20. The fluid and soil monitoring system of claim 17, wherein the sensor device operates without an internal battery through passive wireless power harvesting.
